(19) United States
(12) Patent Application Publication
(10) Pub. No.: US 2002/0193383 A1
(43) Pub. Date: Dec. 19, 2002

(54) 1-(N-phenylalkylaminoalkyl)piperazine derivatives substituted at position 2 of the phenyl ring

(75) Inventors: Amedeo Leonardi, Milano (IT); Gianni Motta, Barlassina (IT); Carlo Riva, Varese (IT); Rodolfo Testa, Vignate (IT)

Correspondence Address: DARBY & DARBY P.C., 805 Third Avenue, New York, NY, 10022, US

(73) Assignee: Recordati S.A., Chemical and Pharmaceutical Comoany

(21) Appl. No.: 10/132,677

(22) Filed: Apr. 22, 2002

Related U.S. Application Data
(63) Continuation-in-part of application No. 09/532,505, filed on Mar. 21, 2000, now Pat. No. 6,399,614.
Application No. 09/532,505 is a continuation-in-part of application No. 09/127,057, filed on Jul. 31, 1998, now Pat. No. 6,071,920.
(60) Provisional application No. 60/070,268, filed on Dec. 31, 1997.

(30) Foreign Application Priority Data
Aug. 1, 1997 (IT) MI97 001864

(51) Int. Cl. A61K 31/496; A61K 31/495; C 07D 4 3/02; C07D 241/04

(52) U.S. Cl. 514/253.13; 514/255.03; 544/360; 544/392

(57) ABSTRACT

The present invention is directed to novel 1-(N-phenylaminoalkyl)piperazine derivatives substituted at the position 2 of the phenyl ring. Pharmaceutical compositions comprising the compounds of the invention also are contemplated. The compounds of the present invention also are contemplated for use in treating neuromuscular dysfunction of the lower urinary tract in a mammal.

### RELATED APPLICATIONS

This application is a continuation-in-part of Ser. No. 09/532,505, filed Mar. 21, 2000, which is a continuation-in-part of Ser. No. 09/127,057, filed Jul. 31, 1998, now U.S. Pat. No. 6,071,920, which claims priority under 35 U.S.C. §119 (e) of U.S. Provisional Patent Application Serial No. 60/070,268, filed Dec. 31, 1997 and priority under 35 U.S.C. §119 (b) of Italian Patent Application No. M197 001864, filed Aug. 1, 1997. Each of the aforementioned applications and patents is hereby incorporated by reference herein in its entirety.

### FIELD OF THE INVENTION

This invention relates to 1-(N-phenylaminoalkyl)piperazine derivatives substituted at position 2 of the phenyl ring, to pharmaceutical compositions containing them and to uses for such derivatives and compositions .

### BACKGROUND OF THE INVENTION

In mammals, micturition (urination) is a complex process that requires the integrated actions of the bladder, its internal and external sphincters, the musculature of the pelvic floor, and neurological control over these muscles at three levels (in the bladder wall or sphincter itself, in the autonomic centers of the spinal cord, and in the central nervous system at the level of the pontine micturition center (PMC) in the brainstem (pons) under the control of cerebral cortex) (De Groat, Neurobiology of Incontinence, (Ciba Foundation Symposium 151:27, 1990). Micturition results from contraction of the detrusor muscle, which consists of interlacing smooth muscle fibers under parasympathetic autonomic control from the sacral spinal cord. A simple voiding reflex is formed by sensory nerves for pain, temperature, and distension that run from the bladder to the sacral cord. However, sensory tracts from the bladder also reach the PMC, resulting in the generation of nerve impulses that normally suppress the sacral spinal reflex arc controlling bladder emptying. Thus, normal micturition is initiated by voluntary suppression of cortical inhibition of the reflex arc and by relaxation of the muscles of the pelvic floor and the external sphincter. Finally, the detrusor muscle contracts and voiding occurs.

Abnormalities of lower urinary tract function, e.g., dysuria, incontinence, and enuresis, are common in the general population. Dysuria includes urinary frequency, nocturia, and urgency, and may be caused by cystitis, prostatitis or benign prostatic hypertrophy (BPH) (which affects about 70% of elderly males), or by neurological disorders. Incontinence syndromes include stress incontinence, urgency incontinence, and overflow incontinence. Enuresis refers to the involuntary passage of urine at night or during sleep.

Prior to the work of the present inventors, treatment of neuromuscular dysfunction of the lower urinary tract has involved administration of compounds that act directly on the bladder muscles, such as flavoxate, a spasmolytic drug (Ruffinan, J. Int. Med. Res. 16:317, 1988) also active on the PMC (Guarneri et al., Drugs of Today 30:91, 1994), or anticholinergic compounds such as oxybutynin (Andersson, Drugs 35:477, 1988). The use of α₁-adrenergic receptor antagonists for the treatment of BPH is also common but is based on a different mechanism of action. (Lepor, Urology, 42:483, 1993).

However, treatments that involve direct inhibition of the pelvic musculature (including the detrusor muscle) may have unwanted side effects such as incomplete voiding or accommodation paralysis, tachycardia and dry mouth (Andersson, Drugs 35:477, 1988). Thus, it would be advantageous if compounds were available that act via the peripheral or central nervous system to, for example, affect the sacral spinal reflex arc and/or the PMC inhibition pathways in a manner that restores normal functioning of the micturition mechanism.

1-(N-phenyl-N-cyclohexylcarbonyl-2-aminoethyl)-4-(2-methoxyphenyl)piperazine (compound A)

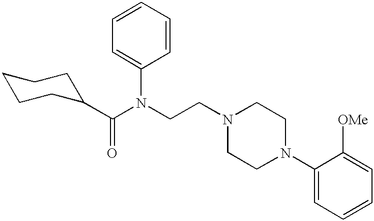

is described in GB 2 263110 A and is reported to be a 5-HT_{1A} receptor antagonist. It is also disclosed that it can be used for the treatment of central nervous system disorders, for example as an anxiolytic agent in the treatment of anxiety.

The compounds of the present invention, described below, are structurally different from compound A because of the novel substituents present on the aniline ring at the 2 position. Other differences between the compounds of the present invention and those disclosed in GB 2 263110 A are the substitutions on the aromatic ring at position 4 of the piperazine ring. These structural variations are neither disclosed nor suggested by GB 2 263110 A, particularly with regard to compounds that can be used to improve urinary tract function. These structural variations result in compounds that are more potent than compound A in pharmacological tests predictive of activity on the lower urinary tract, in particular for activity against urinary incontinence.

Other compounds which have been found by the present inventors to be useful in the methods of the present invention, e.g., treatment of disorders of the urinary tract, are disclosed in U.S. Pat. No. 4,205,173; EP 711,757; DE patent 2,405,441; Chem. Pharm. Bull. 33:1823-1835 (1985), and J. Med. Chem. 7:721-725 (1964), all of which are incorporated by reference.

### SUMMARY OF THE INVENTION

In one aspect the invention is directed to compounds of formula I:

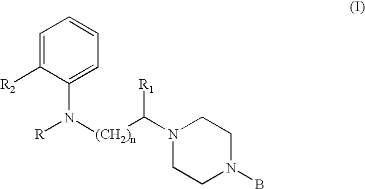

wherein

R is hydrogen

R₁ is chosen from the group consisting of hydrogen and lower alkyl;

R₂ is chosen from the group consisting of alkoxy, phenoxy, nitro, cyano, acyl, amino, acylamino, alkylsulphonylamino, alkoxycarbonyl, N-acylaminocarbonyl, N-alkylaminocarbonyl, N,N-dialkylaminocarbonyl, aminocarbonyl, halo, trifluoromethyl or polyfluroalkoxy group;

n=1 or 2;

B is chosen from the group consisting of optionally substituted aryl, optionally substituted bicyclic aryl group, optionally substituted 9-member bicyclic heteroaromatic containing one heteroatom, and an optionally substituted benzyl group,

with the proviso that if B is aryl and is substituted by an alkoxy group, the alkoxy group must be at the two position; and

if R₁ is hydrogen and R₂ is a nitro group and n=1, then B cannot be a phenyl, 2 methoxyphenyl 4-chlorophenyl, 3 acetylphenyl, 3,4,5 trimethoxyphenyl , 2-chloro-4-methylphenyl or 2-pyridyl group; and

if R and R₁ are hydrogen and B is optionally substituted phenyl, then R₂ cannot be acyl, acylamino, alkoxycarbonyl, N-acylaminocarbonyl, N-alkylaminocarbonyl, N,N-dialkylaminocarbonyl;

and enantiomers N-oxides hydrates and pharmaceutically acceptable salts thereof.

Preferred is when n=1. Further preferred is when B is optionally substituted phenyl. Further preferred is when B is indolyl.

Also preferred is when R₁ is hydrogen; R₂ is chosen from the group consisting of alkoxy, phenoxy, nitro, cyano, amino, halo, trifluoromethyl or polyfluroalkoxy; n=1; and B is substituted phenyl.

Also preferred is when R₁ is hydrogen; R₂ is chosen from the group consisting of alkoxy, phenoxy, nitro, cyano, amino, halo, trifluoromethyl or polyfluroalkoxy; n=1; and B is indolyl.

Also preferred is when R₁ is hydrogen; R₂ is chosen from the group consisting of alkoxy, nitro, halo, trifluoromethyl or polyfluroalkoxy; n=1; and B is substituted phenyl.

Also preferred is when R₁ is hydrogen; R₂ is chosen from the group consisting of alkoxy, nitro, halo, trifluoromethyl or polyfluroalkoxy; n=1; and B is substituted indolyl.

In yet another aspect, the invention is directed to compounds of formula I B

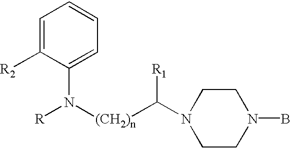

wherein

R is a hydrogen atom, an alkylcarbonyl, a cycloalkylcarbonyl, a substituted cycloalkylcarbonyl or a monocyclic heteroarylcarbonyl group,

n=1 or2;

R₁ is a hydrogen atom or a lower alkyl group,

R₂ is an alkoxy, phenoxy, nitro, cyano, acyl, amino, acylamino, alkylsulphonylamino, alkoxycarbonyl, aminocarbonyl, N-alkylaminocarbonyl, N,N-dialkylaminocarbonyl, N-acylaminocarbonyl, halo, trifluoromethyl or polyfluoroalkoxy group; and

B is a bicyclic heteroaromatic with the proviso that B is not a 9-member bicyclic heteroaromatic containing one heteroatom.

Preferred is when R is chosen from the group consisting of hydrogen and cycloalkylcarbonyl; R₁ is hydrogen; R₂ is chosen from the group consisting of alkoxy, phenoxy, nitro, cyano, acyl, amino, acylamino, alkylsulphonylamino, alkoxycarbonyl, aminocarbonyl, N-alkylaminocarbonyl, N,N-dialkylaminocarbonyl, N-acylaminocarbonyl, halo, trifluromethyl or polyfluroalkoxy group; n=1; and B is a 10-member bicyclic heteroaromatic containing one heteroatom.

Further preferred is when n=1.

Further preferred is when B is a 10-member bicyclic heteroaromatic containing one heteroatom.

Further preferred is when B is an optionally substituted quinolyl.

The invention also includes the enantiomers, diastereomers, N-oxides, crystalline forms, hydrates and pharmaceutically acceptable salts of compounds of the formula 1 and 1B and formula 1C, as well as metabolites of these compounds having the same type of activity (hereafter sometimes referred to as “active metabolites”).

In a preferred aspect are provided compounds of formula 1C

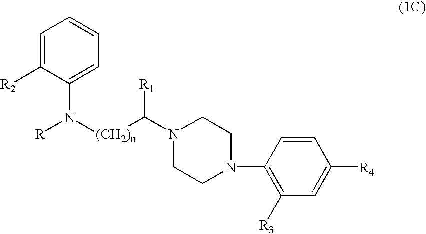

wherein

R=is a hydrogen atom, an alkylcarbonyl, a cycloalkylcarbonyl, a substituted cycloalkylcarbonyl or a monocyclic heteroarylcarbonyl group;

R₁ is chosen from the group consisting of hydrogen and lower alkyl;

R₂ is chosen from the group consisting of alkoxy, phenoxy, nitro, cyano, acyl, amino, acylamino, alkylsulphonylamino, alkoxycarbonyl, N-acylaminocarbonyl, N-alkylaminocarbonyl, N,N-dialkylaminocarbonyl, aminocarbonyl, halo, trifluoromethyl or polyfluroalkoxy group;

R₃ is chosen from the group consisting of methoxy and polyhaloalkoxy;

R₄ is chosen from the group consisting of halogen, hydroxyl, lower alkoxy, lower acyloxy, lower N-alkylaminocarbonyloxy and lower N, N-dialkylaminocarbonyloxy; and

n=1 or2;

and enantiomers, N-oxides, hydrates, and pharmaceutically acceptable salts thereof.

Preferred compounds of formula 1C are where, independently, R is a cycloalkylcarbonyl group, R₁ is a hydrogen atom or lower alkyl group, R₂ is an alkoxy or trifluoromethyl group, R₃ is a methoxy or polyhaloalkoxy group, R₄ is chosen from the group consisting of halogen, hydroxyl, lower alkoxy, lower acyloxy, lower N-alkylaminocarbonyloxy and lower N, N-alkylarninocarbonyloxy, and n=1 or 2. With further respect to compounds of formula 1C, a preferred cycloalkylcarbonyl group for R is cyclohexylcarbonyl group, a preferred alkoxy group for R₂ is methoxy, a preferred polyhaloalkoxy group for R₃ is 2,2,2-trifluoroethoxy, a preferred lower alkoxy group for R₄ is methoxy, preferred lower acyloxy groups for R₄ are acetoxy and 2-methylpropionyloxy, a preferred lower N-alkylaminocarbonyloxy group for R₄ is N-ethylaminocarbonyloxy and the preferred value for n is 1. Each of the foregoing preferences are independent of each other.

Further preferred are compounds of Formula 1C wherein, simultaneously, R is cycloalkylcarbonyl, R₂ is alkoxy or polyfluoroalkoxy, R₃ is polyfluoroalkoxy, R₄ is halogen and n=1 or 2. Also preferred are compounds of Formula 1C wherein, simultaneously, R is cycloalkylcarbonyl, R₂ is polyfluoroalkoxy, R₃ is methoxy, R₄ is selected from the group consisting of hydroxyl, lower alkoxy, lower acyloxy, lower N-alkylaminocarbonyloxy and lower N, N-alkylaminocarbonyloxy groups, and n=1 or 2.

In another aspect, the invention is directed to compounds of formula 1C chosen from the group consisting of:

1-[N-cyclohexylcarbonyl-N-(2-methoxyphenyl)-2-aminoethyl]-4-[4-fluoro-2-(2,2,2-trifluoroethoxy)phenyl]piperazine;

1-[N-cyclohexylcarbonyl-N-(2-trifluoromethoxyphenyl)-2-aminoethyl]-4-[4-fluoro-2-(2,2,2-trifluoroethoxy)phenyl]piperazine;

1-[N-cyclohexylcarbonyl-N-(2-trifluoromethoxyphenyl)-2-aminoethyl]-4-(2,4-dimethoxyphenyl)piperazine;

1 -[N-cyclohexylcarbonyl-N-(2-trifluoromethoxyphenyl)-2-aminoethyl]-4-(4-hydroxy-2-methoxyphenyl)piperazine;

1-[N-cyclohexylcarbonyl-N-(2-trifluoromethoxyphenyl)-2-aminoethyl]-4-(4-acetoxy-2-methoxyphenyl)piperazine;

1-[N-cyclohexylcarbonyl-N-(2-trifluoromethoxyphenyl)-2-aminoethyl]-4-(4-ethylaminocarbonyloxy-2-methoxyphenyl)piperazine; and

1-[N-cyclohexylcarbonyl-N-(2- trifluoromethoxyphenyl)-2-aminoethyl]-4-[4-(2-methylpropionyloxy)-2-methoxyphenyl]piperazine;

and enantiomers, N-oxides, hydrates, and pharmaceutically acceptable salts thereof.

As used herein with reference to variable R, alkylcarbonyl radicals include C₁-C₆ alkylcarbonyl, cycloalkylcarbonyl includes cyclohexylcarbonyl, substituted cycloalkylcarbonyl includes cyclohexylcarbony substituted with alkyl or aryl groups an d monocyclic heteroaryl radicals include monocyclic aromatic radicals of 5 to 7 ring atoms containing one or more hetero atoms (e.g., oxygen, nitrogen, and sulfur). Monocyclic heteroarylcarbonyl has the same definition as monocyclic heteroaryl, but also comprises a carbonyl group linked to a carbon atom of the ring.

As used herein with reference to variable B, a mono or bicyclic aryl radical means an aromatic radical having 6 to 12 carbon atoms (e.g., phenyl or naphthyl) which is substituted by one or more substitutents. Preferred substitutents for aryl radicals are lower alkyl, hydroxy, lower acyloxy (e.g., acetoxy), lower alkylaminocarbonyloxy (e.g., N, ethylaminocarbonyloxy and 2-methylpropionyloxy), lower alkoxy (e.g., methoxy, ethoxy, propoxy, and butoxy), lower haloalkoxy (e.g., 2,2,2-trifluoroethoxy) halogen, amino, acylamino, alkylsulfonylamino, and (lower) alkylamino substituents.

As used with respect to variable B, monocyclic heteroaryl radical has the same meaning as for R, above, and bicyclic heteroaryl radical means a bicyclic aromatic radical containing one or more heteroatoms (e.g., nitrogen, oxygen, sulfur) and 9 to 12 ring atoms. Benzyl radicals, with respect to variable B, include phenylmethyl radicals which may be optionally substituted by one or more substituents. Preferred substituents for the benzyl radicals are alkyl, alkoxy, halogen, nitro, cyano, amido, amino, alkylamino, acylamino, alkylsulphonylamino or acyl substituents. Preferred substituents at B are optionally substituted monocyclic aryl and bicyclic heteroaryl. Most preferred substituents at B are alkoxyphenyl and mononitrogen-containing bicyclic heteroaryl.

Preferred substituents at R₁ are hydrogen and methyl.

Preferred substituents at R₂ are nitro, cyano, acyl, alkoxy, trifluoralkoxy and aminocarbonyl. More preferred at R₂ are nitro, alkoxy and trifluoralkoxy. A preferred value for n is 1.

Preferred substituents for B are optionally substituted phenyl and indolyl.

The invention further provides pharmaceutical compositions comprising a compound of formula I or a compound of formula 1B, or a compound of formula 1C, or an enantiomer, diastereomer, N-oxide, crystalline form, hydrate or pharmaceutically acceptable salt of the compound, in admixture with a pharmaceutically acceptable diluent or carrier.

In another aspect, the present invention is directed to methods for reducing the frequency of bladder contractions due to bladder distension by administering one or more selected compounds of Formula I, or a compound of formula 1B, or a compound of formula 1C, to a mammal (including a human) in need of such treatment, in an amount or amounts effective for the particular use.

In a further aspect, the present invention is directed to methods for treating disorders of the urinary tract in a subject in need of such treatment, comprising administering an effective amount of a compound of Formula 1, or a compound of formula 1B, or a compound of formula 1C, to ameliorate at least one of urinary urgency, increased urinary frequency, incontinence, urine leakage, enuresis, dysuria, urinary hesitancy, and difficulty in emptying bladder.

In yet another aspect, the invention is directed to methods for blocking 5-HT_{1A} serotonergic receptors, and, by virtue of this inhibitory activity, to methods for the treatment of CNS disorders due to serotonergic dysfunction such as anxiety, depression, hypertension, sleep/wake cycle disorders, feeding behavior, sexual function and cognition disorders in mammals, particularly in humans, by delivering to the environment of the 5-HT_{1A} serotonergic receptors, e.g., to the extracellular medium (or by administering to a mammal possessing such receptors) an effective amount of a compound of formula 1, formula 1B or formula 1C (hereinafter “compounds of the invention”).

### DETAILED DESCRIPTION OF THE INVENTION

All patents, patent applications, and literature references cited in the specification are hereby incorporated by reference in their entirety. In the case of inconsistencies, the present disclosure, including definitions, will prevail.

The activity of the compounds of the invention as inhibitors of frequency of micturition renders them useful for the treatment of neuromuscular dysfunctions of the lower urinary tract in mammals, including without limitation dysuria, incontinence and enuresis. Surprisingly, the introduction of selected substituents at position 2 of the aniline ring in compounds of Formula I, Formula IB and Formula 1C confers upon these compounds a distinctly higher potency with regard to compound A and also to the isomers bearing the same substituent at position 3 or 4. This information was obtained by testing compound A and the corresponding 2, 3 and 4 nitroaniline derivatives (Example 2 and compounds B and C) in a rat model. The rhythmic contraction of rat bladders was induced by filling the bladders with a physiologic solution. The effect of test compounds of the invention on the frequency and amplitude of the contractions was evaluated. Of particular interest is the time of disappearance of induced contractions. (ED_{10 min} in Table 1, below).

Data in Table 1 (in particular, ED₅₀ (frequency)) show that the 2-substituted compounds of the invention are clearly more potent inhibitors of the frequency of urinary bladder contractions.

The effect of the drugs currently available for administration to humans for treatment of neuromuscular function of the lower urinary tract (flavoxate and oxybutynin) on the above-described rat model is also shown, for comparative purposes, in Table 1.

The compounds of the invention are more potent and acted for a longer period of time (as measured by duration of bladder quiescence with no contractions) than did compounds A, flavoxate, and oxybutynin. Moreover, in contrast to oxybutynin, the compounds of the invention did not affect the amplitude of the contractions (no effect on ED₅₀ (amplitude) in Table 1), suggesting no impairment of bladder contractility that could result in residual urine being left in the bladder after micturition.

In addition, the beneficial effect on the lower urinary tract of the compounds of the invention has been shown in a cystometry model in conscious rats, where the compounds of the invention are also superior to compound A and the reference drugs. In fact, compounds of the invention increased the bladder capacity at doses lower than compound A and flavoxate (oxybutynin does not affect bladder capacity) (Table 2). Furthermore, in contrast to the effects of oxybutynin, no impairment of bladder contractility (decrease in MP) was observed.

Finally, the compounds of the invention have a high and selective affinity for the 5-HT_{1A} receptor, an affinity and selectivity displayed to a much lesser extent by compound A, (Table 3). The compounds of the invention have also been shown to antagonize both pre- and post-synaptic 5-HT_{1A} receptors much more potently than compound A (Table 4), strongly suggesting a role for the 5-HT_{1A} receptor in the action of the compounds of the invention.

Subjects who can benefit from administration of the compounds and compositions of the invention include humans who are affected by neuromuscular dysfunction of the lower urinary tract, described by E. J. McGuire in “Campbell's UROLOGY” 5^{th} Ed. 616-638, 1986, W. B. Saunders Company, and also include patients affected by any physiological dysfunction related to impairment of 5-HT_{1A} receptor function. Such dysfunctions include, without limitation, central nervous system disorders such as depression, anxiety, eating disorders, sexual dysfunction, addiction, and related problems.

The present invention encompasses pharmaceutical formulations comprising the compounds disclosed above, as well as methods employing these formulations for treating neuromuscular dysfunction of the lower urinary tract such as dysuria, incontinence, enuresis, and the like. Dysuria includes urinary frequency, nocturia, urgency, and difficulty in emptying the bladder, i.e., a suboptimal volume of urine is expelled during micturition.

Incontinence syndromes include stress incontinence, urgency incontinence, and overflow incontinence. Enuresis refers to the involuntary passage of urine at night or during sleep.

Without wishing to be bound by theory, it is believed that administration of the 5-HT_{1A} receptor antagonists of the invention prevents unwanted activity of the sacral reflex arc and/or cortical mechanisms that control micturition. Thus it is contemplated that a wide range of neuromuscular dysfunctions of the lower urinary tract can be treated using the compounds of the present invention.

An “effective amount” of the compound for treating a urinary disorder is an amount that results in measurable amelioration of at least one symptom or parameter of the disorders described above.

An effective amount for treating the disorder can easily be determined by empirical methods known to those of ordinary skill in the art, such as by establishing a matrix of dosages and frequencies of administration and comparing a group of experimental units or subjects to each point in the matrix. The exact amount to be administered to a patient will vary depending on the state and severity of the disorder and the physical condition of the patient. A measurable amelioration of any symptom or parameter can be determined by a physician skilled in the art or reported by the patient to the physician. It will be understood that any clinically or statistically significant attenuation or amelioration of any symptom or parameter of urinary tract disorders is within the scope of the invention. Clinically significant attenuation or amelioration means perceptible to the patient and/or to the physician.

For example, a single patient may suffer from several symptoms of dysuria simultaneously, such as, for example, urgency and excessive frequency of urination, either or both of which may be reduced using the methods of the present invention. In the case of incontinence, any reduction in the frequency or volume of unwanted passage of urine is considered a beneficial effect of the present methods of treatment.

The compounds of the present invention may be formulated into liquid dosage forms with a physiologically acceptable carrier, such as, for example, phosphate buffered saline or deionized water. The pharmaceutical formulation may also contain excipients, including preservatives and stabilizers, that are well-known in the art. The compounds can be formulated into solid oral or non-oral dosage units such as, for example, tablets, capsules, powders, and suppositories, and may additionally include excipients, including without limitation lubricant(s), plasticizer(s), colorant(s), absorption enhancer(s), bactericide(s), and the like.

Modes of administration include oral and enteral, intravenous, intramuscular, subcutaneous, transdermal, transmucosal (including rectal and buccal), and by-inhalation routes. Preferably, an oral or transdermal route is used (i.e., via solid or liquid oral formulations, or skin patches, respectively).

The amount of the agent to be administered can range from between about 0.01 and about 25 mg/kg/day, preferably from between about 0.1 and about 10 mg/kg/day and most preferably from between about 0.2 and about 5 mg/kg/day. It will be understood that the single pharmaceutical formulations of the present invention need not contain the entire amount of the agent that is effective in treating the disorder, as such effective amounts can be reached by administration of a plurality of doses of such pharmaceutical formulations.

In a preferred embodiment of the present invention, compounds are formulated in capsules or tablets, each preferably containing 50-200 mg of the compounds of the invention, and are most preferably administered to a patient at a total daily dose of 50-400 mg, preferably 150-250 mg, and most preferably about 200 mg for relief of urinary incontinence and dysfunctions amenable to treatment with 5-HT_{1A} receptor ligands.

The methods, tables and examples provided below are intended to more fully describe preferred embodiments of the invention and to demonstrate its advantages and applicability, without in any way limiting the scope of the invention.

### SYNTHESIS OF THE COMPOUNDS OF THE INVENTION

The compounds of the invention may be prepared by the methods illustrated in the following reaction schemes, or by modifications thereof, using readily available starting materials, reagents and conventional synthesis procedures well known to those of ordinary skill in the art.

Unless otherwise specified, the substituents of the compounds and intermediates present in the reaction schemes are defined in the same manner as they are defined above in formula I, formula IB and formula 1C. One method to synthesize compounds of formula I and formula IB (R=H) and formula IB and Formula 1C (R=H) is depicted in Scheme 1.

Ortho-substituted anilines of formula II (Y=NH₂) are alkylated with 1,ω-disubstituted alkanes (Z) to give product III. The reaction is carried out in an inert organic solvent, preferentially a polar aprotic solvent such as N,N-dimethylformamide (DMF), dimethylsulfoxide (DMSO), dioxane, tetrahydrofuran (THF), acetone, acetonitrile or chlorinated solvents such as dichloromethane, chloroform, 1,2-dichloroethane or a protic solvent such as n-butanol (n-BuOH). The reactions are generally performed at a temperature between 0° C. and +120° C., in the presence of a proton acceptor such as triethylamine (Et₃N), diisopropylethylamine, or the like, and optionally in the presence of potassium iodide.

In compounds of formula Z, X and X₁ can be Cl, Br, I, aryl, or alkylsulfonyloxy

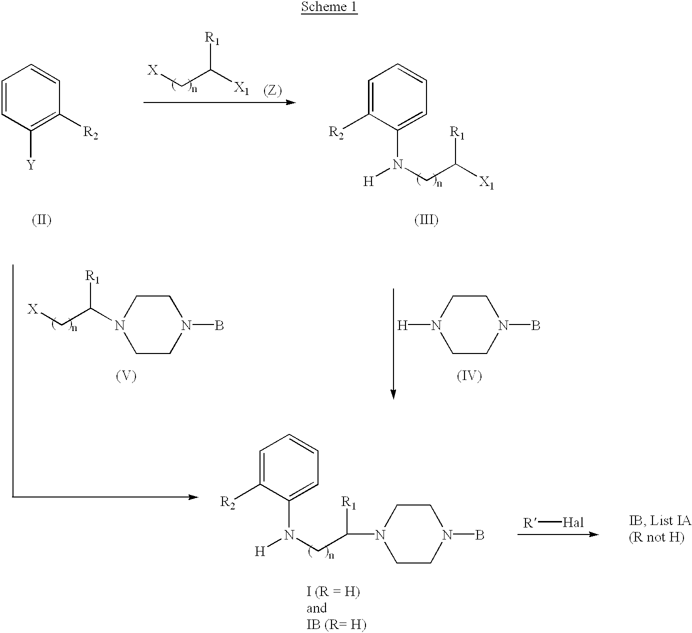

Intermediates of formula III are used in the alkylation of suitable piperazine derivatives IV to give the compounds of formula I and IB (where R=H).

These alkylations may be carried out in a chlorinated solvent such as dichlorometane, chloroform or 1,2-dichloroethane, or in a polar aprotic solvent such as DMF, THF, acetone, acetonitrile, or in a polar protic solvent such as n-BuOH, etc., or in an a polar solvent such as toluene, benzene, n-heptane, etc., at a temperature between 0° C. and 120° C., optionally in the presence of a proton acceptor, such as Et₃N, 4-dimethylaminopyridine, potassium carbonate, cesium carbonate, and the like, and optionally in the presence of potassium iodide.

Piperazines of formula IV which are not commercially available may be prepared by reaction of the suitable B-NH₂ derivatives (which generally may be easily obtained by reduction of the corresponding B-NO₂ derivatives) with bis-(2-chloroethyl)amine or bis-(2-hydroxyethyl)amine in presence of excess hydrogen chloride. These reactions can be performed in aprotic solvents such as dimethylformamide, diglyme or toluene at a temperature between +40° C. and the reflux temperature of the solvent, generally in the presence of a base such as potassium carbonate, cesium carbonate, or the like, and optionally in the presence of potassium iodide.

Compounds of formula V can be conveniently prepared starting from compounds V in which X is a COO-lower alkyl group and n is n-1. Conventional reduction procedures (e.g., use of lithium aluminum hydride or other metal complex hydrides) afford the corresponding compounds V in which X is CH₂OH and n is n-1, which can be in turn conventionally converted into compounds of formula V in which X is a leaving group as described above. The starting esters can be prepared by well known Michael reactions or by the nucleophilic displacement reaction of a monosubstituted piperazine on the appropriate 2,3-unsaturated ester or 2-haloester.

Alternative procedures to obtain compounds of formula V consists in alkylating the appropriate monosubstituted piperazine derivatives with a compound with the formula X—CH(R₁)(CH₂)ₙ₋₁CH₂—OPrG or X—(CH₂)ₙCH(R₁)—X where X is a leaving group and n has the same meaning as above, and PrG is a protecting group (e.g. O-tetrahydropyranyl), which can be removed after alkylation of the piperazine.

Another approach to synthesize intermediate compounds of formula III utilizes starting materials with structure II (Y═halogen). These starting materials are reacted with compounds of formula Z in which X and X₁ are, respectively, NH₂ and OH. These alkylation reactions are carried out in an aprotic solvent such as DMF, toluene, or in a polar protic solvent such as n-BuOH, etc., at a temperature between +40° C. and +140° C., in general using one equivalent or excess of a reagent of formula Z (X═NH₂) as a proton acceptor, as described by G. Doleschall et al., Tetrahedron, 32, 57-64 (1976). The resulting aminoalcohols of formula III (X₁═OH) are reacted with a chlorinating agent such as POCl₃, SOCl₂ or PCl₅ to give the intermediates, also of formula III (X₁═Cl), or with an alkyl or arylsulfonyl chloride to give the corresponding sulfonyl esters. These reactions are carried out in an aprotic solvent such as chloroform, DMF, pyridine, and the like at a temperature between +50° C. and the reflux temperature of the solvent.

Compounds of formula I and IB (R═H) may also be obtained by alkylation of compounds of formula II (Y═NH₂) with intermediates of formula V, in which B, R₁ and n have the same meanings as above and X is a halogen atom such as chlorine or bromine, or a leaving group such as methanesulfonyloxy or p-toluenesulfonyloxy groups.

These reactions may be carried out without solvent or in an aprotic solvent such as dichloromethane, chloroform, DMF, THF, acetone, acetonitrile or in a protic solvent such as n-butanol, etc. at a temperature between 0° C. and +160° C., optionally in the presence of a proton acceptor, such as Et₃N, potassium carbonate, cesium carbonate, 4-dimethylaminopyridine and the like, and optionally in the presence of potassium iodide.

Compounds of formula I and IB where R₂ is CN can be also obtained from the compounds of formula I and IB in which R₂ is CONH₂ by dehydration reactions. P₂O₅, PCl₅, Ph₃P, and the like may be used as dehydrating agents (J. March, Advanced Organic Chemistry, IV Ed., page 1041, Wiley Interscience, 1992). Dehydration reactions may be carried out in a chlorinated solvent such as dichloromethane, chloroform, carbon tetrachloride or in an aprotic solvent such as DMF, toluene, etc. at a temperature between +40° C. and the reflux temperature of the solvent, optionally in the presence of a base such as Et₃N.

Alternatively,compounds of formula I and IB (R═H) may be obtained by arylation of intermediates of formula V (X═NH₂) with a starting material of formula II (Y═Cl, Br, F, I or trifluoromethanesulphonyloxy). These reactions may be carried out using the same solvents and conditions as described above or by employing palladium complex catalysis (Synlett,p.329 (1996)).

Compounds of formula I and IB in which R₂ is COalk can be synthesized from compounds I in which R₂ is H by an acylation reaction that can be carried out using boron trichloride as a Lewis acid and acetonitrile as a reagent in an aprotic solvent such as chloroform, 1,2-dichloroethane, toluene, etc. at temperatures between 0° C. and 100° C., followed by acidic hydrolysis by treatment with HCl at 100° C., (T. Sugasawa et al., Chem. Pharm. Bull., 33, 1826-1835 (1985)).

Compounds of formula I and IB (R═H) are acylated to give compound IB (R other than H) by reaction with an appropriate acyl halide R′Hal in which R′ represents an alkylcarbonyl, cycloalkylcarbonyl or monocyclic heteroarylcarbonyl group and Hal represents a halogen atom. The reaction can be performed in aprotic solvents such as dichloromethane, chloroform, 1,2-dichloroethane, DMF, acetone, acetonitrile, toluene, etc., at temperatures between 0° C. and 100° C., optionally in the presence of an organic base as a proton acceptor such as Et₃N, diisopropylethylamine (DIPEA), 4-dimethylaminopyridine, and the like.

Alternatively,compounds with formula IB (i.e, where R₂═Br, I, OSO₂F or OSO₂CF₃) in which R is as defined above, but is not hydrogen, may be used to synthesize compounds of formula I in which R₂ is CN, CONH₂, COCH₃ or COOCH₃ by reaction of reagents such as trimethylsilyl isocyanate and t-butyl lithium (J. Org. Chem. 55, 3114 (1990)), lithium cyanide and tetrakis(triphenylphosphine)palladium(0) (EP711757), carbon monoxide-methanol and palladium diacetate in the presence of 1,3-diphenylphosphinopropane (J. Org. Chem. 59, 6683 (1994)). Such reactions may be carried out in polar or a polar solvent such as THF, toluene, benzene, DMSO, and the like.

Another method to synthesize compounds of formula I and IB in which R₁ is H is depicted in Scheme 2, below.

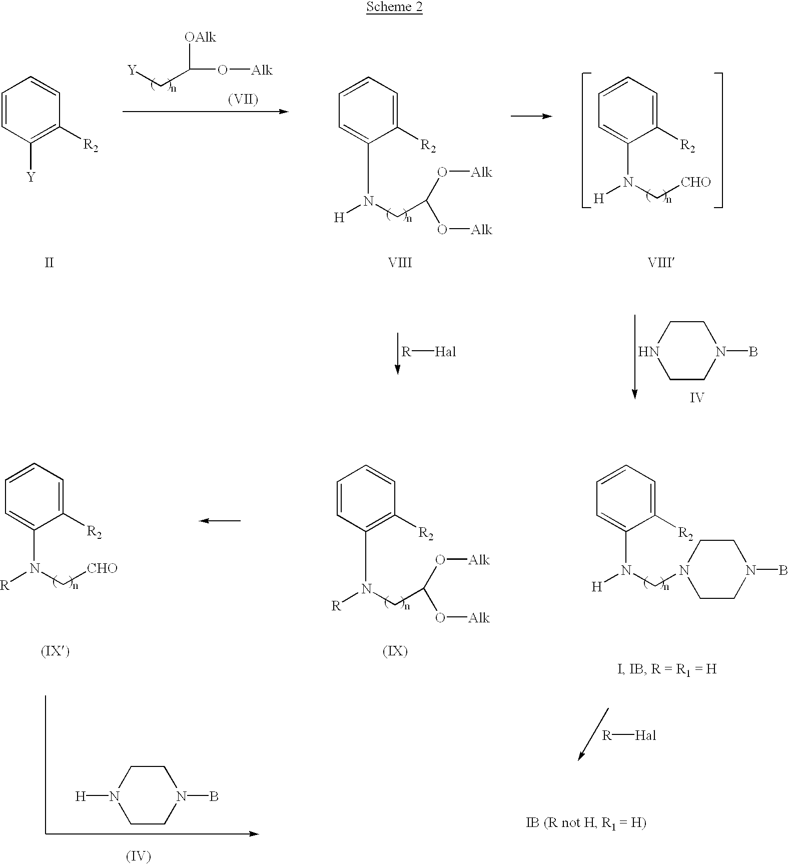

Ortho-substituted halobenzenes of formula II (Y═halo) are used to arylate protected aminoalkylaldehydes of formula VII (X═NH₂) to give the corresponding protected arylaminoalkylaldehydes VIII. The reaction may be carried out in an aprotic solvent such as pyridine, DMF, toluene, or the like at a temperature between +40° C. and 120° C., optionally in the presence of a base such as Et₃N or employing palladium complex catalysts as above.

Another route for the preparation of intermediates of formula VIII consists in alkylating compounds of formula II (Y═NH₂) with protected reactive compounds of formula VII (X═halo) by conventional procedures known to those skilled in the art. Compounds with formula VIII are stable and are deprotected by standard methods just before their use in the following steps.

Aldehydes of formula VIII′, obtained from deprotection of compounds with formula VIII, may be reacted without isolation with N-substituted piperazines IV under reductive conditions to give compounds of formula I and IB (R═R₁═H). These reactions may be carried out in polar solvents such as methanol, ethanol or in chlorinated solvents, such as dichloromethane, chloroform, and the like, using alkali borohydrides such as NaBH₄ and NaBH₃CN, NaBH(OAc)₃ or using borane complexes such as BH₃-Py, optionally in the presence of acidic promoter, such as acetic acid, at temperatures between +10° C. and 100° C.

Alternatively, intermediates of formula VIII may be acylated with R′Hal to give compounds of formula IX using the same conditions as described above.

Intermediates of formula IX are deprotected by well-known methods just before their use in the final step to give the corresponding aldehydes (IX′), which may be reacted with appropriate N-substituted piperazines of formula IV using alkali borohydrides such as NaBH₄, NaBH₃CN or NaBH(OAc)₃, optionally in the presence of catalytic amounts of acetic acid, or of a titanium catalyst such as titanium tetraisopropoxide, yielding compounds of formula I. These reactions may be carried out in chlorinated solvents such as dichloromethane or chloroform, or in polar aprotic solvents such as methanol or ethanol at temperatures between +10° C. and +100° C.

An alternative procedure to afford compounds of formula I, IB and 1C (Scheme 3), where R₂ is an electron withdrawing group (i.e. NO₂, CN, I) consists of acylating by conventional procedure with R′-Hal (R′═alkylcarbonyl) the proper aniline (II, Y═NH₂) to obtain compounds X, which in turn can be alkylated with compounds V where X is a leaving group. The alkylation of compound X can be carried out by preforming the aza-anion of X through the use of bases (e.g. potassium tert-butoxide, NaNH₂, Na, NaH, buthyl lithium or other lithium bases, NaOH/KOH by phase transfer catalysis) in a solvent such as toluene, dimethylsulfoxide, DMF, acetonitrile, acetone, diethyl ether, dioxane, tetrahydrofurane at a temperature included in the range between −25° C. and the temperature of reflux of the solvent. The following alkylation to afford compounds I and IB can be performed by adding to the reaction mixture the compounds V in the same reaction condition as above.

The same alkylation reaction of compounds X can be carried out using compound VII as an alternative methods to prepare compounds IX.

The compounds of formula IB and (R═alkylcarbonyl, R₂═alkylCO) can be prepared from the proper compounds X (R₂═alkylCO) by protecting the keto group (e.g. as 1,3-dioxolanyl derivatives) by standard procedures, then by alkylating the nitrogen of the amido group as described above. Subsequent deprotection affords the desired compounds IB.

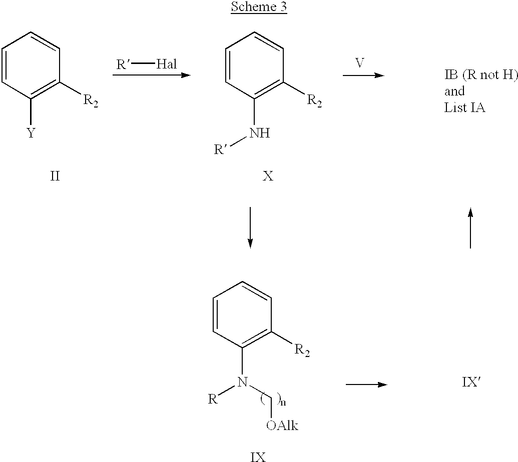

In addition, compounds of formula I where R is alkylcarbonyl, cycloalkylcarbonyl or monocyclic heteroarylcarbonyl group could also be synthesized using this methodology.

Compounds of formula I where B is disubstituted phenyl and one of the substitutions is a lower acyloxy or a lower N-alkylaminocarbonyloxy or lower N, N-dialkylaminocarbonyl-oxy group (i.e., wherein the alkyl groups attached to the nitrogen atom of the respective N-alkylaminocarbonyloxy or N, N-dialkylaminocarbonyloxy groups are lower alkyl groups) can be prepared starting from the corresponding compound of formula I in which one of the two substituents is a hydroxy group. Reactions can be carried out by standard procedures of esterification or addition of a lower alky isocyanate, well known to those skilled in the art.

### Compound AA

1-(N-phenyl-2-aminoethyl)-4-(2-methoxyphenyl)piperazine

### Compounds B and D

1-[N-(3-nitrophenyl)-N-cyclohexylcarbonyl-2-aminoethyl]-4-(2-methoxyphenyl)piperazine (Compound B); and

1-[N-(3-nitrophenyl)-2-aminoethyl]-4-(2-methoxyphenyl)piperazine (Compound D)

### Compounds C and E

1-[N-(4-nitrophenyl)-N-cyclohexylcarbonyl-2-aminoethyl]-4-(2-methoxyphenyl)piperazine (Compound C); and

1-[N-(4-nitrophenyl)-2-aminoethyl]-4-(2-methoxyphenyl)piperazine (Compound E)

Methods for synthesizing Examples 1-89 are described in U.S. patent application Ser. No. 09/532,505, filed Mar. 21, 2000, now U.S. Pat. No. ______, which is hereby incorporated herein in its entirety.

### EXAMPLE 1

1-[N-(2-nitrophenyl)-2-aminoethyl]-4-(2-methoxyphenyl)piperazine

### EXAMPLE 2

1-[N -(2- nitrophenyl)-N-cyclohexylcarbonyl-2-aminoethyl]-4-(2-methoxyphenyl) piperazine

### EXAMPLE 3

1N-(2-trifluoromethoxyphenyl)-2-aminoethyl]-4-(2-methoxyphenyl) piperazine

### EXAMPLE 4

1-[N -(2-trifluoromethoxyphenyl)-N-cyclohexylcarbonyl-2-aminoethyl]-4-(2-methoxyphenyl)piperazine

### EXAMPLE 5

1-[N-(2-phenoxyphenyl)-2-aminoethyl]-4-(2-methoxyphenyl) piperazine

### EXAMPLE 6

1-[N -(2-phenoxyphenyl)-N-cyclohexylcarbonyl-2-aminoethyl]-4-(2-methoxy phenyl)piperazine

### EXAMPLE 7

1-[N-(2-iodophenyl)-2-aminoethyl]-4-(2-methoxyphenyl) piperazine

### EXAMPLE 8

1-[N-(2-iodophenyl)-N-cyclohexylcarbonyl-2-aminoethyl]-4-(2-methoxyphenyl) piperazine

### EXAMPLE 9

1-[N-(2-aminocarbonylphenyl)-2-amininoethyl]-4-(2-methoxyphenyl) piperazine

### EXAMPLE 10

1-[N-(2-cyanophenyl)-2-aminoethyl]-4-(2-methoxyphenyl) piperazine

### EXAMPLE 11

1-[N-(2-acetylphenyl)-2-aminoethyl]-4-(2-methoxyphenyl) piperazine

### EXAMPLE 12

1-[N-(2-nitrophenyl)-2-aminoethyl]-4-(4-indolyl)piperazine

### EXAMPLE 13

1-[N-(2-nitrophenyl)-N-cyclohexylcarbonyl-2-aminoethyl]-4-(4-indolyl) piperazine

### EXAMPLE 14

1-[N-(2-nitrophenyl)-2-aminoethyl)-4-(2,5-dichlorobenzyl) piperazine

### EXAMPLE 15

1-[N-(2-nitrophenyl)-N-cyclohexylcarbonyl-2-aminoethyl]-4-(2,5-dichlorobenzyl) piperazine

### EXAMPLE 16

1- [N-(2-cyclohexylcarbonylaminocarbonylphenyl)-N-cyclohexylcarbonyl-2-amino ethyl]-4-(2-methoxyphenyl)piperazine

### EXAMPLE 17

1-[N-(2-methoxycarbonylphenyl)-2-aminoethyl]-4-(2-methoxyphenyl)piperazine

### EXAMPLE 18

1-[N-(2-methoxycarbonylphenyl)-N-cyclohexylcarbonyl-2-aminoethyl]-4-(2-methoxyphenyl)piperazine

### EXAMPLE 19

1-[N-(2-dimethylaminocarbonylphenyl)-2-aminoethyl]-4-(2-methoxyphenyl) piperazine

### EXAMPLE 20

1-[N-(2-methoxyphenyl)-2-aminoethyl]-4-(2-methoxyphenyl) piperazine

### EXAMPLE 21

1-[N-(2-dimethylaminocarbonylphenyl)-N-cyclohexylcarbonyl-2-aminoethyl]-4-(2-methoxyphenyl)piperazine

### EXAMPLE 22

1-[N-(2-trifluoromethylphenyl)-2-aminoethyl]-4-(2-methoxyphenyl)piperazine

### EXAMPLE 23

1-[N-(2-methoxyphenyl)-N-cyclohexylcarbonyl-2-aminoethyl]-4-(2-methoxy phenyl)piperazine

### EXAMPLE 24

1-[N-(2-ethylaminocarbonylphenyl)-2-aminoethyl]-4-(2-methoxyphenyl)piperazine

### EXAMPLE 25

1-[N-(2-ethylaminocarbonylphenyl)-N-cyclohexylcarbonyl-2-aminoethyl]-4-(2-methoxyphenyl)piperazine

### EXAMPLE 26

1-[N-(2-trifluoromethylphenyl)-N-cyclohexylcarbonyl-2-aminoethyl]-4-(2-methoxyphenyl)piperazine

### Preparation of N-(2-trifluoromethylphenyl cyclohexanecarboxamide (Compound 26A)

A solution of 2-trifluoromethylaniline (3 mL), triethylamine (3.5 mL) and CH₂Cl₂ (30 mL) was stirred at room temperature under N₂ and added dropwise with cyclohexanecarbonyl chloride (3.34 mL). After 2.5 h stirring at room temperature, the mixture was poured into H₂O and alkalinized with 1 N NaOH. The organic phase was dried on anhydrous Na₂SO₄ and the crude was crystallized from EtOH to give 3.82 g (59%) of the title compound. M.p. 153-154° C.

¹H—NMR (CDCl₃, δ): 8.20 (dd, 1H, trifluoromethylphenyl ring CH), 7.60-7.40 (m, 3H, trifluoromethylphenyl ring CHs and NH), 7.12 (ddd, 1H, trifluoromethylphenyl ring CH), 2.30 (tt, 1H, CHC(O)), 2.10-1.20 (m, 10H, cyclohexyl protons).

### Preparation of 1-[N-(2-trifluoromethylphenyl)-N-cyclohexylcarbonl -2-aminoethyl]-4-(2-methoxyphenyl)piperazine

A mixture of N-(2-trifluoromethylphenyl)cyclohexanecarboxamide (0.2 g) (Compd 26A), 1-(2-chloroethyl)-4-(2-methoxyphenyl)piperazine (0.37 g), 50% (w/w) NaOH (0.5 mL), TEBAC (0.16 g) and toluene (2 mL) was stirred at 80° C. for 3.5 h. An additional amount of compd 26A (0.2 g) was then added and after 6 h stirring at 80° C. the mixture was poured into H₂O and extracted with CH₂Cl₂. The organic phase was dried on anhydrous Na₂SO₄, evaporated to dryness and the residue purified by flash chromatography (EtOAc-petroleum ether 3:7) giving 0.12 g (17%) of the title compound.

¹H—NMR (CDCl₃, δ): 7.77 (dd, 1H, trifluoromethylphenyl ring CH), 7.70-7.45 (m, 3H, trifluoromethylphenyl ring CHs), 7.10-6.80 (m, 4H, methoxyphenyl ring CHs), 4.70-4.50 (m, 1H, CONCH(H)CH₂N), 3.85 (s, 3H, OCH₃), 3.20-2.90 (m, 5H, CONCH(H)CH₂N and piperazine protons), 2.85-2.45 (m, 7H, CHC(O), CONCH₂CH₂N and piperazine protons), 1.90-0.75 (m, 10H, cyclohexyl protons).

### EXAMPLE 27

1-[N-(2-aminophenyl)-N-cyclohexylcarbonyl-2-aminoethyl]-4-(2-methoxyphenyl) piperazine

### EXAMPLE 28

1-[N-(2-acetylaminophenyl)-N-cyclohexylcarbonyl-2-aminoethyl]-4-(2-methoxy phenyl)piperazine

### EXAMPLE 29

1-[N-(2-nitrophenyl)-N-cyclohexylcarbonyl-2-aminoethyl]-4-(2-methoxyphenyl) piperazine N¹-oxide

### EXAMPLE 30

1-[N-(2-nitrophenyl)-N-cyclohexylcarbonyl-2-aminoethyl]-4-(2-methoxyphenyl) piperazine N⁴-oxide

### EXAMPLE 31

1-[N-(2-nitrophenyl)-N-cyclohexylcarbonyl-2-aminoethyl]-4-(2-methoxyphenyl) piperazine N¹,N⁴-dioxide

### EXAMPLE 32

1-[N-(2-nitrophenyl)-N-(3-furylcarbonyl)-2-aminoethyl]-4-(2-methoxyphenyl) piperazine

### EXAMPLE 33

1-[N-(2-nitrophenyl)-N-(2-furylcarbonyl)-2-aminoethyl]-4-(2-methoxyphenyl) piperazine

### EXAMPLE 34

1-[N-(2-nitrophenyl)-N-(2-thiophenecarbonyl)-2-aminoethyl]-4-(2-methoxy phenyl)piperazine

### EXAMPLE 35

1-[N-(2-nitrophenyl)-N-(3-thiophenecarbonyl)-2-aminoethyl]-4-(2-methoxy phenyl)piperazine

### EXAMPLE 36

1-[N-(2-nitrophenyl)-N-(4-pyridylcarbonyl)-2-aminoethyl]-4-(2-methoxyphenyl) piperazine

### EXAMPLE 37

1-[N-(2-nitrophenyl)-N-(3-pyridylcarbonyl)-2-aminoethyl]-4-(2-methoxyphenyl) piperazine

### EXAMPLE 38

1-[N-(2-nitrophenyl)-N-(2-pyrazinylcarbonyl)-2-aminoethyl]-4-(2-methoxy phenyl)piperazine

### EXAMPLE 39

1-[N-(2-nitrophenyl)-N-(1-methylcyclohexylcarbonyl)-2-aminoethyl]-4-(2-methoxy phenyl)piperazine

### EXAMPLE 40

1-[N-(2-nitrophenyl)-N-(1-phenylcyclohexylcarbonyl)-2-aminoethyl]-4-(2-methoxyphenyl)piperazine

### EXAMPLE 41

1-[N-[2-(2,2,2-trifluoroethoxy)phenyl]-2-aminoethyl]-4-(2-methoxyphenyl) piperazine

### EXAMPLE 42

1-[N-[2-(2,2,2-trifluoroethoxy)phenyl]-N-cyclohexylcarbonyl-2-aminoethyl]-4-92-methoxyphenyl)piperazine

### EXAMPLE 43

1-[N-(2-cyanophenyl)-N-cyclohexylcarbonyl-2-aminoethyl]-4-(2-methoxy phenyl)piperazine hydrochloride

### EXAMPLE 44

1-[N-(2-nitrophenyl)-1-amino-2-propyl]-4-(2-methoxyphenyl)piperazine

### EXAMPLE 45

1-[N-(2-nitropheny)-N-cyclohexylcarbonyl-1-amino-2-propyl]-4-(2-methoxyphenyl)piperazine

### EXAMPLE 46

1-[N-cyclohexylcarbonyl-N-(2-methanesulphonylaminophenyl)-2-aminoethyl]-4-(2-methoxyphenyl)piperazine

### EXAMPLE 47

1-[N-(2-methoxyphenyl)-2-aminoethyl]-4-(4-indolyl)piperazine

### EXAMPLE 48

1-[N-cyclohexylcarbonyl-N-(2-methoxyphenyl)-2-aminoethyl]-4-(4-indolyl)piperazine

### EXAMPLE 49

1-[N-cyclohexylcarbonyl-N-(2-benzyloxycarbonylphenyl)-2-aminoethyl]-4-(2-methoxyphenyl)piperazine

### EXAMPLE 50

1-[N-(2-trifluoromethoxypheny l)-2-aminoethyl]-4-(4-indolyl) piperazine

### EXAMPLE 51

1-[N-cyclohexylcarbonyl-N-(2-trifluoromethoxyphenyl)-2-aminoethyl]-4-(4-indolyl)piperazine

### EXAMPLE 52

1-[N-(2-nitrophenyl)-2-aminopropyl]-4-(2-methoxyphenyl)piperazine

### EXAMPLE 53

1-[N-(2-nitrophenyl)-N-(2-pyrazinecarbonyl)-2-aminoethyl)]-4-(4-indolyl)piperazine

### EXAMPLE 54

1-[N-(2-trifluoromethoxyphenyl)-2-aminoethyl]-4-(2-methoxy-4-nitrophenyl)-piperazine

### EXAMPLE 55

1-[N-cyclohexylcarbonyl-N-(2-trifluoromethoxyphenyl)-2-aminoethyl]-4-(2-methoxy-4-nitrophenyl)piperazine

### EXAMPLE 56

1-[N-cyclohexylcarbonyl-N-(2-trifluoromethoxyphenyl)-2-aminoethyl]-4-(4-amino-2-methoxyphenyl)piperazine

### EXAMPLE 57

1-[N-cyclohexylcarbonyl-N-(2-trifluoromethoxyphenyl)-2-aminoethyl]-4-(4-acetylamino-2-methoxyphenyl)piperazine

### EXAMPLE 58

1-[N-cyclohexylcarbonyl-N-(2-trifluoromethoxyphenyl)-2-aminoethyl]-4-(4-trifluoroacetylamino-2-methoxyphenyl)piperazine

### EXAMPLE 59

1-[N-cyclohexylcarbonyl-N-(2-propoxycarbonylphenyl)-2-aminoethyl]-4-(2-methoxyphenyl)piperazine

### EXAMPLE 60

1-[N-cyclohexylcarbonyl-N-(2-nitrophenyl)-2-aminoethyl]-4-[4-(N-acetyl-N-cyclohexylcarbonyl)amino-2-methoxyphenyl]piperazine

### EXAMPLE 61

1-[N-cyclohexylcarbonyl-N-(2-nitrophenyl)-2-aminoethyl]-4-[4-(bis-cyclohexylcarbonyl)amino-2-methoxyphenyl]piperazine

### EXAMPLE 62

1-[N-cyclohexylcarbonyl-N-(2-nitrophenyl)-2-aminoethyl]-4-[4-cyclohexanecarbonylamino-2-methoxyphenyl)piperazine

### EXAMPLE 63

1-[N-(2-nitrophenyl)-N-(2-pyrimidinecarbonyl)-2-aminoethyl)]-4-(2-methoxyphenyl)piperazine

### EXAMPLE 64

1-[N-[2-(2,2,2-trifluoroethoxy)phenyl)]-2-aminoethyl]-4-(2-methoxy-4-nitrophenyl)piperazine

### EXAMPLE 65

1-[N-cyclohexylcarbonyl-N-[2-(2,2,2-trifluoroethoxyphenyl)]-2-aminoethyl)]-4-(2-methoxy-4-nitrophenyl)piperazine

### EXAMPLE 66

1-[N-cyclohexylcarbonyl-N-[2-(2,2,2-trifluoroethoxyphenyl)]-2-aminoethyl)]-4-(4-amino-2-methoxyphenyl)piperazine

### EXAMPLE 67

1-[N-cyclohexylcarbonyl-N-[2-(2,2,2-trifluoroethoxyphenyl)]-2-aminoethyl)]-4-(4-acetylamino-2-methoxyphenyl)piperazine

### EXAMPLE 68

1-[N-[2-(2,2,2-trifluoroethoxy)phenyl)]-2-aminoethyl]-4-(4-indolyl)piperazine

### EXAMPLE 69

1-[N-cyclohexylcarbonyl-N-[2-(2,2,2-trifluoroethoxyphenyl)]-2-aminoethyl]-4-(4-indolyl)piperazine

### EXAMPLE 70

1-[N-cyclohexylcarbonyl-N-(2-nitrophenyl)-2-aminoethyl)]-4-(4-acetylamino-2-methoxyphenyl)piperazine

### EXAMPLE 71

1-[N-(2-trifluoromethoxyphenyl)-2-aminoethyl]-4-(2-trifluoromethoxy phenyl)piperazine

### EXAMPLE 72

1-[N-cyclohexylcarbonyl-N-(2-trifluoromethoxyphenyl)-2-aminoethyl]-4-(2-trifluoromethoxyphenyl)piperazine

### EXAMPLE 73

1-[N-(2-nitrophenyl)-N-(5-thiazolylcarbonyl)-2-aminoethyl)]-4-(2-methoxyphenyl)piperazine

### EXAMPLE 74

1-[N-(2-trifluoromethoxyphenyl)-2-aminoethyl]-4-(2-bromo-5-methoxybenzyl)piperazine

### EXAMPLE 75

1-[N-cyclohexylcarbonyl-N-(2-trifluoromethoxyphenyl)-2-aminoethyl]-4-(2-bromo-5-methoxybenzyl)piperazine

### EXAMPLE 76

1-[N-cyclohexylcarbonyl-N-(2-iodophenyl)-2-aminoethyl]-4-(4-indolyl)piperazine

### EXAMPLE 77

1-[N-(2-cyanophenyl)-N-cyclohexylcarbonyl-2-aminoethyl]-4-(2-trifluoromethoxyphenyl)piperazine

### EXAMPLE 78

1-[N-(2-cyanophenyl)-N-cyclohexylcarbonyl-2-aminoethyl]-4-(2-methoxy-4-nitrophenyl)piperazine

### EXAMPLE 79

1-[N-(2-cyanophenyl)-N-cyclohexylcarbonyl-2-aminoethyl]-4-(4-amino-2-methoxyphenyl)piperazine

### EXAMPLE 80

1-[N-(2-cyanophenyl)-N-cyclohexylcarbonyl-2-aminoethyl]-4-(4-acetylamino-2-methoxyphenyl)piperazine

### EXAMPLE 81

1-[N-(2-acetylphenyl)-N-cyclohexylcarbonyl-2-aminoethyl]-4-(2-methoxyphenyl)piperazine

### EXAMPLE 82

1-[N-(2-cyanophenyl)-N-cyclohexylcarbonyl-2-aminoethyl]-4-(4-indolyl)piperazine

### EXAMPLE 83

1-[N-(2-cyanophenyl)-N-cyclohexylcarbonyl-2-aminoethyl]-4-(2-bromo-5-methoxybenzyl)piperazine

### EXAMPLE 84

1-[N-cyclohexylcarbonyl-N-(2-trifluoromethoxyphenyl)-2-aminoethyl]-4-(1 -cyclohexylcarbonyl-4-indolyl)piperazine

### EXAMPLE 85

1-[N-cyclohexylcarbonyl-N-(2-trifluoromethoxyphenyl)-2-aminoethyl]-4-(8-quinolyl)piperazine

### Preparation of N-(2-trifluoromethoxyphenyl)cyclohexanecarboxamide (Compound 85A)

This compound was prepared following the procedure described for compound 26A of example 26, except that 2-trifluoromethoxyaniline was used in place of 2-trifluoromethylaniline. Yield: 100%.

¹H—NMR (CDCl₃, δ): 8.44 (dd, 1H, phenyl ring H6), 7.47 (bs, 1H, CONH), 7.21-730 (m, 2H, phenyl ring H4, H5), 7.08-7.14 (m, 1H, phenyl ring H3), 2.21-2.43 (m, 1H, CHCO), 1.20-2.08 (m, 10H, cyclohexyl protons).

### Preparation of N-(2 ,2-dimethoxyethyl)-N-(2-trifluoromethoxyphenyl)cyclohexanecarbox-amide (85B)

A solution of compound 85A (0.15 g) in toluene (20 mL) was heated to reflux and 5 mL of toluene distilled off; then t-BuOK (0.09 g) was added and the mixture heated to reflux removing by evaporation 2 mL of the solvent; then 2-bromoacetaldehyde dimethyl acetal (0.09 mL) was dropped. The resulting suspension was refluxed under N₂ atmosphere for 12-15 h. After cooling to room temperature, the solvent was evaporated to dryness in vacuo and the crude purified by flash chromatography (petroleum ether—EtOAc 9:1), affording 0.106 g (54%) of the title compound as an oil.

¹H—NMR (CDCl₃, δ): 7.31-7.48 (m, 4H, phenyl ring CHs), 4.61-4.70 (m, 1H, CH(OCH₃)₂), 4.16-4.24 (m, 1H, CONCH(H)), 3.39 (s, 3H, OCH₃),3.32 (s, 3H, OCH₃), 3.13-3.24 (m, 1H, CONCH(H)), 1.92-2.09 (m, 1H, CHCO), 0.85-1.79 (m, 10H, cyclohexyl protons).

### Preparation of N-(2-oxoethyl)-N-(2-trifluoromethoxyphenyl)cyclohexanecarboxamide (85C)

A suspension of compound 85B (0.106 g), hydroquinone (0.003 g) in 2 N HCl (1.14 mL) was heated at 80° C. for 30-40 minutes. After cooling to room temperature, NaHCO₃ was added (pH=7) and the resulting mixture was extracted with CH₂Cl₂ (3×10 mL).The combined organic layers were dried (Na₂SO₄), filtered and evaporated to dryness at reduced pressure to give 0.081 g (88%) of the title compound as an oil.

¹H—NMR (CDCl₃, δ): 9.62 (s, 1H, CHO), 7.29-7.57 (m, 4H, phenyl ring CHs), 4.67 (d, 1H, NCH(H)CHO), 3.86 (d, 1H, NCH(H)CHO), 2.02-1.98 (m, 1H, CHCO), 0.85-1.83 (m, 10H, cyclohexyl protons).

### Preparation of 1-(8-quinolyl]piperazine (85D)

A mixture of 8-aminoquinoline (1.5 g), bis-(2-chloroethyl)amine hydrochloride (2.04 g), o-dichlorobenzene (4.5 mL) and n-hexanol (0.45 mL) was stirred at reflux temperature for 5 h. After cooling to room temperature, the mixture was treated with 2 N NaOH (10 mL) and extracted with CH₂Cl₂ (3×20 mL). The purification was carried out by flash chromatography (CH₂Cl₂-2 N methanolic NH₃) 97:3 to give 0.48 g (22%) of the title compound.

¹H—NMR (CDCl₃, δ): 8.80 (dd, 1H, quinolyl H2), 8.17 (dd, 1H, quinolyl H4), 7.32-7.53 (m, 3H, quinolyl H6, H5, H3), 7.12 (m, 1H, quinolyl H7), 3.38-3.51 (m, 4H, piperazine protons), 3.21-3.32 (m, 4H, piperazine protons), 2.21 (bs, 1H, NH).

### Preparation of 1-[N-cyclohexylcarbonyl-N-(2-trifluoromethoxyphenyl)-2-aminoethyl]-4-(8-quinolyl)piperazine

To a solution of compound 85C (0.081 g), compound 85D (0.06 g) and acetic acid (0.06 mL) in 1,2-DCE (10 mL) stirred under N₂ atmosphere, NaB(Oac)₃H (0.078 g) was added. The resulting mixture was stirred for 2 h at 20-25° C., alkalinized with 1 N NaOH (pH=8) and extracted with CH₂Cl₂ (3×10 mL). The organic layer was dried over Na₂SO₄, filtered and evaporated at reduced pressure. The crude was purified by flash chromatography (toluene-acetone 75:25) to give 0.09 g (73%) of the title compound. M.p. 141.5-143° C.

¹H—NMR (CDCl₃, δ): 8.86 (dd, 1H, quinolyl H2), 8.14 (dd, 1H, quinol H4), 7.30-7.52 (m, 7H, quinolyl H3, H5, H6 and phenyl ring CHs), 7.09-7.18 (m, 1H, quinolyl H7), 4.32-4.47 (m, 1H, CONCH(H)CH₂N), 3.21-3.51 (m, 5H, piperazine protons and CONCH(H)CH₂N), 2.53-2.94 (m, 6H, piperazine protons and CONCH₂CH₂N), 1.89-2.07 (m, 1H, CHCO), 0.81-1.79 (m, 10H, cyclohexyl protons).

### EXAMPLE 86

1-[N-cyclohexylcarbonyl-N-(2-trifluoromethoxyphenyl )-2-aminoethyl]-4-(4-amino-2-cyanophenyl)piperazine

### EXAMPLE 87

1-[N-cyclohexylcarbonyl-N-(2-trifluoromethoxyphenyl )-2-aminoethyl]-4-(2-cyanophenyl)piperazine

### EXAMPLE 88

1-[N-cyclohexylcarbonyl-N-(2-methanesulphonylaminophenyl)-2-aminoethyl]-4-(4-indolyl)piperazine

### EXAMPLE 89

1-[N-cyclohexylcarbonyl-N-(2-methoxyphenyl)-2-aminoethyl]-4-(1-cyclohexylcarbonyl-4-indolyl)piperazine

### EXAMPLE 90

1-[N-cyclohexylcarbonyl-N-(2-methoxyphenyl)-2-aminoethyl]-4-[4-fluoro-2-(2,2,2-trifluoroethoxy)phenyl]piperazine

### Preparation of [2-(2-methoxyphenyl)amino]acetaldehyde dimethyl acetal (Compound 90A)

A stirred mixture of 5.8 mL of 2-methoxyaniline, 50 mL of anhydrous DMF, 20.75 g of anhydrous K₂CO₃ and 15.5 mL of 97% 2-bromoacetaldehyde dimethyl acetal was heated at 150° C. for 5 h. After diluting with H₂O (500 mL), the mixture was extracted with Et₂O (4×70 mL); the organic layer was washed with H₂O (5×50 mL), dried (anhydrous Na₂SO₄) and evaporated to dryness in vacuo. The residue was purified by flash chromatography (petroleum ether-EtOAc 90:10) to afford 9.5 g (90.5%) of Compond 90A as a yellow oil.

¹H—NMR (CDCl₃, δ): 3.26 (d, 2H, CH₂), 3.43 (s, 6H, 2 CH₃O), 3.85 (s, 3H, CH₃O), 4.00-4.70 (br, 1H, NH), 4.61 (t, 1H, CH), 6.56-6.95 (m, 4H, phenyl CHs) After D₂O treatment the NH signal appeared as HDO peak.

### Preparation of N-(2 ,2-dimethoxyethyl)-N-(2-methoxyphenyl)cyclohexanecarboxamide (Compound 90B)

A stirred mixture of 0.73 g of Compound 90A, 40 mL of CH₂Cl₂, 1 mL of 97% Et₃N and 0.60 mL (4.4 mmol) of 98% cyclohexanecarbonyl chloride was maintained at room temperature for 24 h, washed with H₂O (5×20 mL), 2 N NaOH (6×20 mL), dried (anhydrous Na₂SO₄) and evaporated to dryness in vacuo. The residue was purified by flash chromatography (petroleum ether-EtOAc 80:20) to afford 0.955 g (86%) of Compound 90B as an oil.

¹H—NMR (CDCl₃,δ): 0.75-1.75 (m, 10H, cyclohexane CH₂s), 1.90-2.15 (m, 1H, CHCO), 3.18 (dd, 1H, CONCH(H)CH), 3.25 and 3.35 (2s, 6H, 2 CH₃O), 3.81 (s,3H, CH₃O), 4.20 (dd, 1H, CONCH(H)CH), 4.60(ddd, 1H, CONCH(H)CH), 6.85-7.05 (m, 2H, phenyl CHs), 7.15-7.40 (m, 2H, phenyl CHs)

### Preparation of N-(2-oxoethyl)-N-(2-methoxyphenyl)cyclohexanecarboxamide (Compound 90C)

A stirred mixture of 0.955 g of Compound 90B, 0.10 g of hydroquinone and 20 mL of 2 N HCl was heated to 80° C. for 30′ under nitrogen atmosphere. After cooling at 0-5° C., 60 mL of CH₂Cl₂ was added and the mixture was alkalinized with 20% aq. Na2CO₃. The layers were separated and the alkaline one was re-extracted with CH₂Cl₂ (2×40 mL). The combined organic layers were dried (anhydrous Na₂SO₄) and evaporated to dryness in vacuo to afford 0.646 g (79%) of Compound 90C as an orange oil.

¹H—NMR (CDCl₃, δ): 0.80-1.85 (m, 10H, cyclohexane CH₂s), 2.10-2.30 (m, 1H, CHCO), 3.82 (s, 3H, CH₃O), 4.18 (d, 2H, CH₂), 6.90-7.05 (m, 2H, phenyl CHs), 7.20-7.45(m, 2H, phenyl CHs), 9.65 (s, 1H, CHO)

### Preparation of 1-[N-cyclohexylcarbonl-N-(2-methoxyphenyl)-2-aminoethyl]-4-[4-fluoro-2-(2,2 ,2-trifluoroethoxy)phenyl]piperazine

The title compound was prepared following the method described in example 85 substituting compound 90C for compound 85C and 1-[4-fluoro-2-(2,2,2-trifluoroethoxy)phenyl]piperazine for 1-(8-quinolyl)piperazine. The residue was purified by flash chromatography (CHCl₃-2 N methanolic ammonia 100:1) to afford the title compound g (57%) as a yellow oil.

¹H—NMR (CDCl₃, δ): 0.80-1.75 (m, 1OH, cyclohexane CH₂s), 1.90-2.12 (m, 1H, CHCO), 2.40-2.75 (m, 6H, CONCH(H)CH₂, piperazine CHs), 2.85-3.10 (m, 4H, piperazine CHs), 3.30-3.50 (m, 1H, CONCH(H)CH₂), 3.82 (s, 3H, CH₃O), 4.08-4.28 (m, 1H,CONCH(H) CH₂), 4.38 (q, 2H, OCH₂CF₃), 6.58-6.80(m, 2H, fluorophenyl H3 and H6), 6.80-7.05 (m, 3H, fluorophenyl H5, methoxyphenyl CHs), 7.15-7.43 (m, 2H, methoxyphenyl CHs)

### EXAMPLE 91

1-[N-cyclohexylcarbonyl-N-(2-trifluoromethoxyphenyl)-2-aminoethyl]-4-[4-fluoro-2-(2,2,2-trifluoroethoxy)phenyl]piperazine

The title compound was prepared following the method described in example 85 substituting 1-[4-fluoro-2-(2,2,2-trifluoroethoxy)phenyl]piperazine for 1-(8-quinolyl)piperzine. The residue was purified by flash chromatography (CHCl₃-2 N methanolic ammonia 100:1) to afford the title compound (90%) as a yellow oil.

¹H—NMR (CDCl₃, δ): 0.75-1.78 (m, 10H, cyclohexane CH₂s), 1.80-2.08 (m, 1H, CHCO), 2.40-2.75 (m, 6H, CONCH(H)CH₂, piperazine CHs), 2.85-3.10 (m, 4H, piperazine CHs), 3.18-3.40 (m, 1H, CONCH(H)CH₂), 4.20-4.48 (m, 3H, CF₃CH₂O, CONCH(H)CH₂), 6.58-6.80 (m, 2H, fluorophenyl H3 and H6), 6.80-6.97 (m, 1H, fluorophenyl H5), 7.21-7.50 (m, 4H, trifluoromethoxyphenyl CHs)

### EXAMPLE 92

1-[N-cyclohexylcarbonyl-N-(2-trifluoromethoxyphenyl)-2-aminoethyl]-4-(2,4-dimethoxyphenyl)piperazine

The title compound was prepared following the method described in example 85 substituting 1-(2,4-dimethoxyphenyl)piperazine for 1-(8-quinolyl)piperazine. The residue was purified by flash chromatography (CH₂Cl₂-2 N methanolic ammonia 100:1) to afford the title compound (80%) as an ivory oil.

¹H—NMR (CDCl₃, δ): 0.85-1.78 (m, 10H, cyclohexane CH₂s), 1.78-2.05 (m, 1H, CHCO), 2.40-2.75 (m, 6H, CONCH(H)CH₂, piperazine CHs), 2.80-3.05 (m, 4H, piperazine CHs), 3.15-3.40 (m, 1H, CONCH(H)CH₂), 3.75 and 3.80 (s, 6H, 2 CH₃O), 4.20-4.40 (m, 1H, CONCH(H)CH₂), 6.32-6.50 (m, 2H, methoxyphenyl H3 and H5), 6.80 (d, 1H, methoxyphenyl H6), 7.20-7.50 (m, 4H, trifluoromethoxyphenyl CHs)

### EXAMPLE 93

1[N-cyclohexylcarbonyl-N-(2-trifluoromethoxyphenyl)-2-aminoethyl]-4-(4-hydroxy-2-methoxyphenyl)piperazine

The title compound was prepared following the method described in example 85 substituting 1-(4-hydroxy-2-methoxyphenyl)piperazine.2H₂O for 1-(8-quinolyl)piperazine. The residue was purified by flash chromatography (CHCl₃-2 N methanolic ammonia 100:2) to afford the title compound (55%) as an ivory amorphous solid.

¹H—NMR (CDCl₃, δ): 0.75-1.75 (m, 10H, cyclohexane CH₂s), 1.82-2.08 (m, 1H, CHCO), 2.40-2.75 (m, 6H, CONCH(H)CH₂, piperazine CHs), 2.75-3.05 (m, 4H, piperazine CHs), 3.15-3.40 (m, 1H, CONCH(H)CH₂), 3.80 (s, 6H, CH₃O), 4.20-4.40 (m, 1H, CONCH(H)CH₂), 4.65-5.50 (br, 1H, OH), 6.32 (dd, 1H, methoxyphenyl H5), 6.42 (d, 1H, methoxyphenyl H3), 6.78 (d, 1H, methoxyphenyl H6), 7.20-7.50 (m, 4H, trifluoromethoxy-phenyl CHs)

### EXAMPLE 94

1-[N-cyclohexylcarbonyl-N-(2-trifluoromethoxyphenyl)-2-aminoethyl]-4-(4-acetoxy-2-methoxyphenyl)piperazine

To a mixture of 0.15 g of the compound of Example 93, 20 mL of CH₂Cl₂ and 0.06 mL of 97% Et₃N was added 0.03 mL of 98% CH₃COCl at r.t. After 6 h at r.t. the reaction solution was diluted with CH₂Cl₂ (20 mL), washed with H₂O (5×10 mL), 1 N NaOH (2×10 mL), H₂O (2×10 mL), dried (anhydrous Na₂SO₄) and evaporated to dryness in vacuo. The residue was purified by flash chromatography (CH₂Cl₂ -2 N methanolic ammonia 100:3) to afford 0.12 g (73.7%) of the title compound as a yellow oil.

¹H—NMR (CDCl₃, δ): 0.85-1.80 (m, 10H, cyclohexane CH₂s), 1.80-2.05 (m, 1H, CHCO), 2.28 (s, 3H, CH₃COO), 2.40-2.80 (m, 6H, CONCH(H)CH₂, piperazine CHs), 2.80-3.15 (m, 4H, piperazine CHs), 3.15-3.40 (m, 1H, CONCH(H)CH₂), 3.80 (s, 3H, CH₃O), 4.10-4.60 (m, 1H, CONCH(H)CH₂), 6.50-6.70 (m, 2H, methoxyphenyl H3 and H5), 6.85 (d, 1H, methoxyphenyl H6), 7.20-7.50 (m, 4H, trifluoromethoxyphenyl CHs)

### EXAMPLE 95

1-[N-cyclohexylcarbonyl-N-(2-trifluoromethoxyphenyl)-2-aminoethyl]-4-(4-ethylaminocarbonyloxy-2-methoxyphenyl)piperazine

To a stirred solution of 0.24 g (0.46 mmol) of the compound of example 93 in 20 mL of anhydrous THF was added 0.02 g (0.50 mmol) of 60% NaH at r.t. under nitrogen. After stirring for 1 h at r.t., 0.05 mL (0.62 mmol) of 98% ethyl isocyanate was added and the mixture was stirred for 4 h, diluted with H₂O (50 mL) and brine (10 mL) and extracted with EtOAc (3×30 mL). The organic layer was dried (anhydrous Na₂SO₄) and evaporated to dryness in vacuo. The residue was purified by flash chromatography (CHCl₂-2 N methanolic ammonia 100:2) to afford 0.15 g (54.4%) of the title compound as a vitreous ivory solid.

¹H—NMR (CDCl₃, δ): 0.75-1.80 (m, 13H, cyclohexane CH₂s, CH₃CH₂N), 1.80-2.08 (m, 1H, CHCO), 2.40-2.80 (m, 6H, CONCH(H)CH₂, piperazine CHs), 2.80-3.15 (m, 4H, piperazine CHs), 3.15-3.45 (m, 1H, CONCH(H)CH₂), 3.80 (s, 3H, CH₃O), 3.90-4.10 (m, 1H, CH₃CH₂N), 4.20-4.40 (m, 1H, CONCH(H)CH₂), 6.50-6.75 (m, 2H, methoxyphenyl H3 and H5), 6.90 (d, 1H, methoxyphenyl H6), 7.20-7.50 (m, 4H, trifluoromethoxyphenyl CHs), 8.20-8.58 (m, 1H, CONH)

### EXAMPLE 96

1-[N-cyclohexylcarbonyl-N-(2-trifluoromethoxyphenyl)-2-aminoethyl]-4-[4-(2-methylpropionyloxy)-2-methoxyphenyl]piperazine

The title compound was prepared following the procedure described for compound 94 but using 2-methylpropionyl chloride instead of acetyl chloride. The residue was purified by flash chromatography (EtOAc-petroleum ether 1:1) to afford the title compound (17%) as a yellow oil.

¹H—NMR (CDCl₃, δ): 0.75-1.75 (m, 16H, cyclohexane CH₂s, CH(CH₃)₂), 1.80-2.05 (m, 1H, CHCO), 2.35-2.85 (m, 7H, CONCH(H)CH₂, piperazine CHs, CH(CH₃)₂), 2.85-3.10 (m, 4H, piperazine CHs), 3.15-3.40 (m, 1H, CONCH(H)CH₂), 3.80 (s, 3H, CH₃O), 4.20-4.40 (m, 1H, CONCH(H)CH₂), 6.50-6.70 (m, 2H, methoxyphenyl H3 and H5), 6.85 (d, 1H, methoxyphenyl H6), 7.20-7.50 (m, 4H, trifluoromethoxyphenyl CHs).

### EXAMPLE 97

### Effects on Volume-Induced Rhythmic Bladder Voiding Contractions in Anaesthetized Rats

### A. Methods

Female Sprague Dawley rats weighing 225-275 g (Crl: CDo BR, Charles River Italia) were used. The animals were housed with free access to food and water and were maintained on a forced 12 h alternating light-dark cycle at 22-24° C. for at least one week, except during the experiment. The activity on the rhythmic bladder voiding contractions was evaluated according to the method of Dray (J. Pharmacol. Methods, 13:157, 1985), with some modifications as in Guarneri (Pharmacol. Res., 27:173, 1993). Briefly, rats were anesthetized by subcutaneous injection of 1.25 g/kg (5 ml/kg) urethane, after which the urinary bladder was catheterized via the urethra using PE 50 polyethylene tubing filled with physiological saline. The catheter was tied in place with a ligature around the external urethral orifice and was connected with conventional pressure transducers (Statham P23 ID/P23 XL). The intravesical pressure was displayed continuously on a chart recorder (Battaglia Rangoni KV 135 with DCl/TI amplifier). The bladder was then filled via the recording catheter by incremental volumes of warm (37° C.) saline until reflex bladder voiding contractions occurred (usually 0.8-1.5 ml). For intravenous (i.v.) injection of bioactive compounds, PE 50 polyethylene tubing filled with physiological saline was inserted into the jugular vein.

From the cystometrogram, the number of contractions recorded 15 min before (basal values) and after treatment, as well as the mean amplitude of these contractions (mean height of the peaks in mmHg) was evaluated. Since most compounds produced an effect that was relatively rapid in onset and led to a complete cessation of bladder contractions, bioactivity was conveniently estimated by measuring the duration of bladder quiescence (i.e., the duration of time during which no contractions occurred). The number of animals tested showing a reduction in the number of contractions >30% of that observed in the basal period was also recorded.

To compare the potency of the tested compounds for inhibiting bladder voiding contractions, equieffective doses which resulted in a contraction disappearance time of 10 minutes (ED₁₀ₘᵢₙ) were computed by means of least square linear regression analysis. Also computed in this manner were extrapolated doses which induced a reduction of the number of contractions of greater than 30% in 50% of treated rats (ED₅₀, frequency) by the method of Bliss (Bliss C. I., Quart. J. Pharm. Pharmacol. 11, 192-216, 1938). After the suppressive effects of drug injection wore off, the height of the contractile peaks was compared with the height of the peaks previously recorded after the control intravenous administration of vehicle. The potency of the tested compounds (ED₅₀ value: the extrapolated doses inducing a 30% reduction of amplitude of the contractions in 50% of treated rats) was evaluated on a quantal basis by the method of Bliss (Bliss C. I., Quart. J. Pharm. Pharmacol. 11, 192-216, 1938).

### B. Results

The rapid distension of the urinary bladder in urethane-anesthetized rats produced a series of rhythmic bladder voiding contractions whose characteristics have been described and are well-known in the art (Maggi et al., Brain Res., 380:83, 1986; Maggi, et al., J. Pharmacol. Exp. Ther., 230:500, 1984). The frequency of these contractions is related to the sensory afferent arm of reflex micturition and to the integrity of the micturition center, while their amplitude is a property of the efferent arm of the reflex. In this model system, compounds that act mainly on the CNS (such as morphine) cause a block in voiding contraction, whereas drugs that act at the level of the detrusor muscle, such as oxybutynin, lower the amplitude of the bladder contractions.

The results obtained after administration of prior art compounds and compounds of the invention are shown in Table 1. Compound A, a prior art compound, was more potent than flavoxate and oxybutynin in inhibiting voiding contractions. This compound, in contrast to oxybutynin, did not affect the amplitude of the contraction, indicating no impairment of bladder contractility. Surprisingly, however, compounds with substituents at position 2 of the aniline ring in Formula I, such as NO₂, such as the compound of Example 2, have significantly higher potency than unsubstituted compound A, particular with regard to the ED₁₀ₘᵢₙ values. Like compound A, the compound of Example 2 does not affect bladder contractility. When compounds were synthesized with a nitro group at position 3 or 4 of the aniline ring, such as comparative compounds B and C, pharmacological activity was abolished.

Results similar (i.e., higher potency for the 2-substituted derivatives) to those obtained by Example 2 were obtained relative to compounds where R is H and which are unsubstituted or substituted at the 3- and 4-positions of the aniline ring. These results are shown in Table 1, where it can be seen that compounds AA, D, E, which are unsubstituted and 3- and 4-substituted compounds, are clearly inferior to the compounds of Examples 1, 10 and 11 and 18, i.e., 2-NO_{2,} 2-CN, 2-COCH₃ and 2-COOCH₃ derivatives. The compounds of the invention were clearly superior, particularly with regard to the ED₅₀ values which are indicators of urinary frequency.

TABLE 1
Effects on rhythmic bladder voiding
contractions after intravenous administration.
Data represent the ED_{10 min} values (the extrapolated dose
inducing 10 min of disappearance of the contractions); the
ED₅₀ values (the extrapolated doses inducing a reduction of
the number of contractions >30% in 50% of treated rats)
(frequency), and the ED₅₀ values (the extrapolated doses
inducing 30% reduction of amplitude of the contractions in
50% of treated rats) (amplitude).

| | | ED50 | ED50 |
|---|---|---|---|
| Compound | ED_{10 min} μg/kg | (frequency) μg/kg | (amplitude) μg/kg |
| Compound A | 650 | 33 | n.a. |
| Compound B | >1000 | >1000 | n.a. |
| Compound C | >1000 | >1000 | n.a. |
| Compound D | >1000 | >1000 | n.a. |
| Compound E | >1000 | >1000 | n.a. |
| Compound AA | 663 | 244 | n.a. |
| Example 1 | 192 | 55 | n.a. |
| Example 2 | 60 | 9 | n.a. |
| Example 10 | 122 | 28 | n.a. |
| Example 11 | 318 | 40 | n.a. |
| Example 13 | 266 | 29 | n.a. |
| Example 18 | 101 | 17 | n.a. |
| Example 20 | 97 | 25 | n.a. |
| Example 23 | 93 | 18 | n.a. |
| Example 27 | 131 | 13 | n.a. |
| Example 42 | 33 | 18 | n.a. |
| Example 43 | 52 | 4 | n.a. |
| Example 47 | 181 | 20 | n.a. |
| Example 48 | 40 | 14 | n.a. |
| Example 51 | 276 | 200 | n.a. |
| Example 56 | 61 | 21 | n.a. |
| Example 57 | 254 | 252 | n.a. |
| Example 66 | 167 | 58 | n.a. |
| Example 69 | 60 | 14 | n.a. |
| Flavoxate | >10000 | 2648 | n.a. |
| Oxybutinin | 7770 | >10000 | 240 |

n.a. = not active; no significant reduction of the height of peaks.
Compound AA = 1-(N-phenyl-2-aminoethyl)-4-(2-methoxyphenyl)piperazine.
Compounds A, B, C, D, E, see text.

### EXAMPLE 98

### Effects on Cystometric Parameters in Conscious Rats

### A. Methods

Male Sprague Dawley rats (Crl: CDo BR) weighing 250-350 g were used. The animals were housed with free access to food and water and maintained on a forced 12 h alternating light-dark cycle at 22-24° C. for at least one week, except during performance of the experiment. To quantify urodynamic parameters in conscious rats, cystometrographic studies were performed using procedures previously described (Guarneri et al., Pharmacol. Res., 24:175, 1991). Male rats were anesthetized with nembutal (30 mg/kg) and chloral hydrate (125 mg/kg) i.p. and were placed in a supine position. An approximately 10 mm long midline incision was made in the shaved and cleaned abdominal wall. The urinary bladder was gently freed from adhering tissues, emptied, and then cannulated, via an incision at the dome, with a polyethylene cannula (Portex PP30), which was permanently sutured with silk thread. The cannula was exteriorized through a subcutaneous tunnel in the retroscapular area, where it was connected with a plastic adapter to avoid the risk of removal by the animal. For intravenous (i.v.) injection of test compounds, a PE 50 polyethylene tubing filled with physiological saline was inserted into the jugular vein and exteriorized in the retroscapular area. The rats were utilized exclusively one day after implantation. On the day of the experiment, the rats were placed in Bollman's cages; after a stabilization period of 20 min, and the free tip of the bladder catheter was connected through a T-shaped tube to a pressure transducer (Bentley T 800/Marb P 82) and to a peristaltic pump (Gilson minipuls 2) for a continuous infusion, at the constant rate of 0.1 ml/mmin, of saline solution into the urinary bladder. The intraluminal pressure signal during infusion was continuously recorded on a polygraph (Battaglia Rangoni KO 380 with ADCl/T amplifier). Two urodynamic parameters were evaluated: bladder volume capacity (BVC) and micturition pressure (MP). BVC (in ml) is defined as the minimum volume infused after which detrusor contraction (followed by micturition) occurs. MP (in mm Hg) is defined as the maximal intravesical pressure induced by the contraction of detrusor during micturition. Basal BVC and MP values were calculated as the means of the first two recorded cystometrograms. At this point in the assay, the infusion was interrupted and the test compounds were administered. Fifteen minutes after intravenous administration two additional cystometrograms were recorded in each animal and the mean values of the two cystometrographic parameters were calculated. The statistical significance of the differences in urodynamic parameter values was evaluated by Student's t test for paired data.

### B. Results

The effects of different doses of the tested compounds are shown in Table 2. Compound A behaved similarly to flavoxate by increasing BVC. Neither compound impaired bladder contractility, since no consistent changes in MP were observed. In contrast, oxybutynin markedly and dose-dependently decreased MP without effects on BVC. The compound of Example 2 was more potent than compound A and flavoxate; a significant increase in BVC was observed after the i.v. administration of 0.3 mg/kg of the compound of Example 2, compared with the requirement for administration of 1.0 mg/kg of flavoxate or compound A. The compound of Example 2 induced a slight, albeit significant, decrease in MP. This effect, however, was not dose-dependent and was markedly lower than that induced by oxybutynin.

TABLE 2
Effects on cystometrogram in conscious rats.
Data represent mean values ± S.E. of bladder volume
capacity (BVC; ml) and of micturition pressure (MP; mmHg),
before and 15 min after i.v. injection of the compounds.

| | Dose | BVC | | % of |
|---|---|---|---|---|
| COMPOUND | μg/kg | before | after treatment | change |
| Compound A | 300 | 0.81 ± 0.05 | 0.87 ± 0.05 | +7.4 |
| | 1000 | 0.78 ± 0.11 | 0.97 ± 0.11** | +24.4 |
| Example 2 | 300 | 0.71 ± 0.09 | 0.87 ± 0.10* | +22.5 |
| | 1000 | 0.62 ± 0.09 | 0.75 ± 0.10** | +21.0 |
| Example 8 | 300 | 0.59 ± 0.04 | 0.71 ± 0.05* | +21.0 |
| | 1000 | 0.65 ± 0.10 | 0.88 ± 0.12** | +35.0 |
| Example 13 | 100 | 0.94 ± 0.12 | 1.07 ± 0.14 | +13.4 |
| | 300 | 0.73 ± 0.09 | 0.95 ± 0.12** | +30.2 |
| Example 18 | 100 | 0.60 ± 0.07 | 0.80 ± 0.09* | +33.3 |
| | 1000 | 0.63 ± 0.11 | 0.89 ± 0.16** | +40.3 |
| Example 23 | 300 | 0.50 ± 0.06 | 0.77 ± 0.03** | +54.3 |
| | 1000 | 0.66 ± 0.09 | 0.89 ± 0.12** | +34.5 |
| Example 42 | 300 | 0.70 ± 0.11 | 0.89 ± 0.15 | +26.4 |
| | 1000 | 0.70 ± 0.09 | 1.00 ± 0.16* | +41.7 |
| Example 51 | 300 | 0.66 ± 0.11 | 0.84 ± 0.14** | +27.7 |
| | 1000 | 0.79 ± 0.05 | 1.08 ± 0.09** | +36.5 |
| FLAVOXATE | 300 | 0.76 ± 0.11 | 0.87 ± 0.11 | +14.5 |
| | 1000 | 0.88 ± 0.15 | 1.11 ± 0.16** | +26.1 |
| OXYBUTYNIN | 100 | 0.82 ± 0.15 | 0.89 ± 0.18 | +8.5 |
| | 300 | 0.83 ± 0.13 | 0.83 ± 0.12 | ±0.0 |
| | 1000 | 0.94 ± 0.19 | 1.00 ± 0.18 | ±6.4 |
| | Dose | MP | | % of |
| COMPOUND | μg/kg | before | after treatment | change |
| Compound A | 300 | 90.6 ± 10.4 | 85.6 ± 11.3 | −5.5 |
| | 1000 | 90.2 ± 6.5 | 84.1 ± 5.2 | −6.8 |
| Example 2 | 300 | 95.4 ± 6.4 | 80.4 ± 6.5** | −15.7 |
| | 1000 | 109.0 ± 12.1 | 99.6 ± 11.2* | −8.6 |
| Example 8 | 300 | 116.1 ± 17.4 | 98.3 ± 17.2** | −15.0 |
| | 1000 | 81.3 ± 9.0 | 64.8 ± 10.5* | −20.0 |
| Example 13 | 100 | 85.7 ± 14.2 | 75.6 ± 13.7 | −12.5 |
| | 300 | 73.4 ± 11.8 | 65.7 ± 13.8 | −10.6 |
| Example 18 | 100 | 73.9 ± 7.9 | 48.5 ± 4.9** | −34.3 |
| | 1000 | 91.7 ± 13.8 | 79.3 ± 17.0 | −13.5 |
| Example 23 | 300 | 93.5 ± 7.0 | 86.4 ± 10.0 | −7.6 |
| | 1000 | 74.6 ± 10.2 | 61.9 ± 8.5** | −17.1 |
| Example 42 | 300 | 83.1 ± 11.5 | 66.5 ± 9.9** | −20.0 |
| | 1000 | 77.4 ± 5.4 | 70.3 ± 7.8 | −9.2 |
| Example 51 | 300 | 80.0 ± 6.2 | 73.7 ± 6.1 | −7.9 |
| | 1000 | 78.3 ± 6.7 | 67.9 ± 5.6* | −13.3 |
| FLAVOXATE | 300 | 89.2 ± 10.7 | 95.0 ± 10.9 | +6.5 |
| | 1000 | 90.4 ± 10.7 | 80.1 ± 11.1 | −11.4 |
| OXYBUTYNIN | 100 | 95.2 ± 9.2 | 77.4 ± 10.3** | −18.7 |
| | 300 | 82.3 ± 8.7 | 50.5 ± 6.3** | −38.6 |

*= P < 0.05,
**= P < 0.01 versus basal values;
Student's t test for paired data

### EXAMPLE 99

### Binding to 5-HT_{1A} and Other Different Neurotransmitter Binding Sites

### A. Methods

### Recombinant Human 5HT 1A Receptors

Genomic clone G-21 coding for the human 5-HT1A serotonergic receptor is stably transfected in a human cell line (HeLa). HeLa cells were grown as monolayers in Dulbecco's modified Eagle's medium (DMEM), supplemented with 10% fetal calf serum and gentamicin (100 mg/ml), 5% CO₂ at 37° C. Cells were detached from the growth flask at 95% confluence by a cell scraper and were lysed in ice-cold 5 mM Tris and 5 mM EDTA buffer (pH 7.4). Homogenates were centrifuged at 40000×g×20 min and pellets were resuspended in a small volume of ice-cold 5 mM Tris and 5 mM EDTA buffer (pH 7.4) and immediately frozen and stored at −70° C. until use. On the day of experiment, cell membranes were resuspended in binding buffer: 50 mM Tris HCl (pH 7.4), 2.5 mM MgCl2, 10 μM pargiline (Fargin et al., Nature 335, 358-360, 1988). Membranes were incubated in a final volume of 1 ml for 30 min at 30° C. with 0.2-1 nM [3H]8-OH-DPAT, in absence or presence of competing drugs; non-specific binding was determined in the presence of 10 μM 5-HT. The incubation was stopped by addition of ice-cold Tris-HCl buffer and rapid filtration through 0.2% polyethyleneimine pretreated Whatman GF/B or Schleicher & Schuell GF52 filters.

### Native 5-HT_{2A} Serotoninergic Receptors and α₂-adrenoceptors (from Animal Tissues)

Binding studies on native α₂ adrenergic receptors (Diop L. et al, J. Neurochem. 41, 710-715, 1983), and 5-HT2A serotonergic receptors (Craig A. and Kenneth J., Life Sci. 38, 117-127, 1986) were carried out in membranes of rat cerebral cortex. Male Sprague Dawley rats (200-300 g, SD Harlan/Nossan, Italy) were killed by cervical dislocation and cerebral cortexes were excised and immediately frozen in liquid nitrogen and stored at −70° C. until use. Tissues were homogenized (2×20 sec) in 50 volumes of cold 50 mM Tris-HCl buffer pH 7.4, using a Polytron homogenizer (speed 7). Homogenates were centrifuged at 49000×g for 10 min, resuspended in 50 volumes of the same buffer, incubated at 37° C. for 15 min and centrifuged and resuspended twice more. The final pellets were suspended in 100 volumes of 50 mM Tris-HCl buffer pH 7.4, containing 10 μM pargiline and 0.1% ascorbic acid (a2 adrenergic receptors) or in 100 volumes of 50 mM Tris-HCl buffer pH 7.7 (5-HT2A serotonergic receptors). Membranes were incubated in a final volume of 1 ml for 30 min at 25° C. with 0.5-1.5 nM [3H]rauwolscine (a2-adrenergic receptors) or for 20 min at 37° C. with 0.7-1.3 nM [3H]ketanserin (5-HT2A receptors), in absence or presence of competing drugs. Non-specific binding was determined in the presence of 10 μM phentolamine (a2-adrenergic receptors) or 2 μM ketanserin (5-HT2A serotoninergic receptors). The incubation was stopped by addition of ice-cold 50 mM Tris-HCl buffer and rapid filtration through 0.2% polyethyleneimine pretreated Whatman GF/B or Schleicher & Schuell GF52 filters. The filters are then washed with ice-cold buffer and the radioactivity retained on the filters was counted by liquid scintillation spectrometry.

### B. Results

The inhibition of specific binding of the radioligands by the tested drugs was analyzed to estimate the IC50 value by using the non-linear curve-fitting program Allfit (De Lean et al., Am. J. Physiol. 235, E97-E102, 1978). The IC50 value was converted to an affinity constant (Ki) by the equation of Cheng & Prusoff (Cheng, Y. C.; Prusoff, W. H. Biochem. Pharmacol. 22, 3099-3108, 1973).

The results shown in Table 3A demonstrate that compound A and the compound of Example 2 both have a very high affinity for 5-HT_{1A} receptors, but their binding profile is different. The compound of Example 2 was much more selective than compound A for the 5-HT1A receptor versus the 5-HT₂A and the a2-adrenoceptors. All the other compounds of the invention tested (Table 3B) had high affinity for the 5-HT1A receptor.

TABLE 3B
Binding affinity for the 5-HT_{1A} receptor
Data are expressed as Ki (nM).

| Compound | 5-HT_{1A} |
|---|---|
| Ex. 3 | 10.28 |
| Ex. 4 | 0.64 |
| Ex. 5 | 14.85 |
| Ex. 6 | 0.45 |
| Ex. 7 | 3.82 |
| Ex. 8 | 0.36 |
| Ex. 10 | 17.23 |
| Ex. 11 | 2.92 |
| Ex. 12 | 4.77 |
| Ex. 13 | 0.50 |
| Ex. 14 | 10.32 |
| Ex. 15 | 6.20 |
| Ex. 16 | 2.9 |
| Ex. 17 | 20.15 |
| Ex. 18 | 0.60 |
| Ex. 20 | 24.62 |
| Ex. 21 | 2.72 |
| Ex. 22 | 18.18 |
| Ex. 23 | 0.14 |
| Ex. 25 | 8.91 |
| Ex. 26 | 2.69 |
| Ex. 27 | 0.57 |
| Ex. 28 | 18.78 |
| Ex. 30 | 7.96 |
| Ex. 32 | 19.36 |
| Ex. 34 | 16.27 |
| Ex. 35 | 8.00 |
| Ex. 38 | 1.02 |
| Ex. 39 | 2.65 |
| Ex. 45 | 2.21 |
| Ex. 46 | 6.05 |
| Ex. 47 | 2.33 |
| Ex. 48 | 0.46 |
| Ex. 49 | 0.17 |
| Ex. 50 | 1.16 |
| Ex. 51 | 0.24 |
| Ex. 52 | 34 |
| Ex. 53 | 11.75 |
| Ex. 55 | 10.78 |
| Ex. 56 | 2.13 |
| Ex. 57 | 0.74 |
| Ex. 58 | 2.44 |
| Ex. 65 | 18.62 |
| Ex. 66 | 0.76 |
| Ex. 67 | 4.76 |
| Ex. 68 | 9.32 |
| Ex. 69 | 0.32 |
| Ex. 70 | 28.09 |
| Ex. 71 | 15.48 |
| Ex. 72 | 1.22 |
| Ex. 73 | 21.23 |
| Ex. 74 | 2.02 |
| Ex. 76 | 0.69 |
| Ex. 77 | 0.58 |
| Ex. 83 | 1.31 |
| Ex. 84 | 23.23 |
| Ex. 88 | 1.03 |
| Ex. 93 | 1.60 |
| Ex. 94 | 2.14 |
| Ex. 96 | 1.20 |

### Measurement of Pre- and Post-Synaptic 5-HT_{1A} Receptor Antagonist Activity

### A. Methods

### Antagonism of Hypothermia Induced by 8-OH-DPAT in Mice (Pre-synaptic Antagonism).

The antagonistic effect of the ⁵-HT_{1A} receptor antagonists of the invention on hypothermia induced by 8-OH-DPAT was evaluated by the method of Moser (Moser, Eur. J. Pharmacol., 193:165, 1991) with minor modifications as described below. Male CD-1 mice (28-38 g) obtained from Charles River (Italy) were housed in a climate-controlled room (temperature 22±2 C.; humidity 55±15%) and maintained on a 12 h light/dark cycle with free access to food and water. On the day of experiment, mice were placed singly in clear plastic boxes under the same ambient conditions. Body temperature was measured by the insertion of a temperature probe (Termist TM-S, LSI) into the rectum to a depth of 2 cm. Rectal temperature was measured immediately prior to intravenous injection of the test compound. All animals then received 8-OH-DPAT (0.5 mg/kg s.c.) and their temperature was measured 30 min later. For each animal, temperature changes were calculated with respect to pretreatment values and the mean values were calculated for each treatment group. A linear regression equation was used in order to evaluate ID₅₀ values, defined as the dose of antagonist needed to block 50% of the hypothermic effect induced by 0.5 mg/kg 8-OH-DPAT administered subcutaneously.

### Inhibition Offorepaw Treading Induced by 8-OH-DPA Tin Rats (Post-synaptic Antagonism)

The inhibitory effect of 5-HT_{1A} receptor antagonists on the forepaw treading induced in rats by subcutaneous injection of 8-OH-DPAT was evaluated by the method of Tricklebank (Tricklebank et al., Eur. J. Pharmacol., 117:15, 1985) with minor modifications as described below.

Male Sprague-Dawley rats (150-175 g) obtained from Charles River (Italy), were housed in a climate-controlled room and maintained on a 12 h light/dark cycle with free access to food and water. On the day of experiment, rats were placed singly in clear plastic boxes. Rats were treated with reserpine, 1 mg/kg s.c., 18-24 h before the test to deplete intracellular stores of noradrenaline. For evaluation of antagonistic activity, compounds were i.v. administered 16 min before 8-OH-DPAT (1 mg/kg s.c.). Observation sessions of 30 s duration began 3 min after treatment with the agonist and were repeated every 3 min over a period of 15 min. The appearance of the forepaw treading symptom induced by postsynaptic stimulation of the 5HT_{1A} receptors was noted, and its intensity was scored using a ranked intensity scale in which: 0=absent, 1=equivocal, 2=present and 3=intense. Behavioral scores for each treated rat were accumulated over the time course (5 observation periods) and expressed as mean values of 8-10 rats. A linear regression equation was used in order to evaluate ID₅₀ values, defined as the dose of antagonist needed to block 50% of the forepaw treading intensity induced by 1 mg/kg 8-OH-DPAT administered subcutaneously.

### B. Results

The results are shown in Table 4. These results demonstrate that compound of Example 2 exhibits significant pre-synaptic and post-synaptic 5-HT_{1A} receptor antagonist activity. Compound A, by contrast, proved at least 10 fold less active than compound of Example 2 in both models.

TABLE 4
Antagonistic activity for the pre- and post-synaptic 5-HT_{1A} receptor.
Data are expressed as ID₅₀ in mg/kg.

| | Pre-synaptic 5-HT_{1A} | Post-synaptic 5-HT_{1A} |
|---|---|---|
| Compound | ID₅₀ | ID₅₀ |
| Compound A | 221 | 350 |
| Example 2 | 20 | 36 |
| Example 13 | — | 82 |
| Example 18 | n.a. | 84 |
| Example 23 | — | 177 |

What is claimed:

1. A compound of the formula 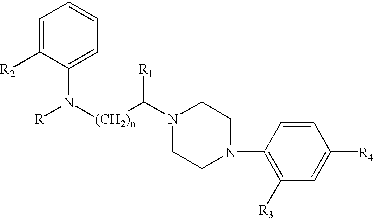
wherein
R=is a hydrogen atom, an alkylcarbonyl, a cycloalkylcarbonyl, a substituted cycloalkylcarbonyl or a monocyclic heteroarylcarbonyl group;
R₁ is chosen from the group consisting of hydrogen atom and lower alkyl group;
R₂ is chosen from the group consisting of halogen atom, alkoxy, phenoxy, nitro, cyano, acyl, amino, acylamino, alkylsulphonylamino, alkoxycarbonyl, N-acylaminocarbonyl, N-alkylaminocarbonyl, N,N-dialkylaminocarbonyl, aminocarbonyl, trifluoromethyl and polyfluroalkoxy groups;
R₃ is chosen from the group consisting of methoxy and polyhaloalkoxy groups;
R₄ is chosen from the group consisting of halogen aton, hydroxyl, lower alkoxy, lower acyloxy, lower N-alkylaminocarbonyloxy and lower N, N-dialkylaminocarbonyloxy groups; and
n=1 or 2;
or an enantiomer, N-oxide, hydrate, or pharmaceutically acceptable salt thereof.

2. A compound according to claim 1 wherein n=1.

3. A compound according to claim 1 wherein R is cycloalkylcarbonyl.

4. A compound according to claim 3 wherein R is cyclohexylcarbonyl.

5. A compound according to claim 1 wherein R₂ is selected from the group consisting of alkoxy and polyfluroalkoxy groups.

6. A compound according to claim 5 wherein R₂ is selected from the group consisting of methoxy and trifluoromethoxy groups.

7. A compound according to claim 4 wherein R₂ is selected from the group consisting of methoxy and trifluoromethoxy groups.

8. A compound according to claim 1 wherein R₃ is polyhaloalkoxy and R₄ is halogen.

9. A compound according to claim 8 wherein R is cycloalkylcarbonyl.

10. A compound according to claim 9 wherein R is cyclohexylcarbonyl.

11. A compound according to claim 8 wherein R₂ is selected from the group consisting of alkoxy and polyfluroalkoxy groups.

12. A compound according to claim 1 wherein R₄ is chosen from the group consisting of hydroxyl, lower alkoxy, lower acyloxy, lower N-alkylaminocarbonyloxy and lower N, N-dialkylaminocarbonyloxy groups.

13. A compound according to claim 12 wherein R₄ is chosen from the group consisting of lower acyloxy, lower N-alkylaminocarbonyloxy and lower N, N-dialkylaminocarbonyloxy groups.

14. A compound according to claim 13 wherein R is cycloalkylcarbonyl.

15. A compound according to claim 14 wherein R is cyclohexylcarbonyl.

16. A compound according to claim 15 wherein R₂ is a polyfluoroalkoxy group.

17. A compound according to claim 16 wherein R₂ is a trifluoromethoxy group.

18. A pharmaceutical composition comprising a compound of claim 1 and a pharmaceutically acceptable diluent, excipient or carrier.

19. The pharmaceutical composition of claim 18 which comprises at least one excipient selected from the group consisting of lubricants, plasticizers, colorants, absorption enhancers, and bactericides.

20. A compound selected from the group consisting of:
1-[N-cyclohexylcarbonyl-N-(2-methoxyphenyl)-2-aminoethyl]-4-[4-fluoro-2-(2,2,2-trifluoroethoxy)phenyl]piperazine;
1-[N-cyclohexylcarbonyl-N-(2-trifluoromethoxyphenyl)-2-aminoethyl]-4-[4-fluoro-2-(2,2,2-trifluoroethoxy)phenyl]piperazine;
1-[N-cyclohexylcarbonyl-N-(2-trifluoromethoxyphenyl)-2-aminoethyl]-4-(2,4-dimethoxyphenyl)piperazine;
1-[N-cyclohexylcarbonyl-N-(2-trifluoromethoxyphenyl)-2-aminoethyl]-4-(4-hydroxy-2-methoxyphenyl)piperazine;
1-[N-cyclohexylcarbonyl-N-(2-trifluoromethoxyphenyl)-2-aminoethyl]-4-(4-acetoxy-2-methoxyphenyl)piperazine;
1 -[N-cyclohexylcarbonyl-N-(2-trifluoromethoxyphenyl)-2-aminoethyl]-4-(4-ethylaminocarbonyloxy-2-methoxyphenyl)piperazine; and
1-[N-cyclohexylcarbonyl-N-(2-trifluoromethoxyphenyl)-2-aminoethyl]-4-[4-(2-methylpropionyloxy)-2-methoxyphenyl]piperazine;
and enantiomers, N-oxides, hydrates, and pharmaceutically acceptable salts thereof.

21. A pharmaceutical composition comprising a compound of claim 20 and a pharmaceutically acceptable diluent, excipient or carrier.

22. The pharmaceutical composition of claim 21 which comprises at least one excipient selected from the group consisting of lubricants, plasticizers, colorants, absorption enhancers, and bactericides.

23. A method for treating neuromuscular dysfunction of the lower urinary tract in a mammal in need of such treatment, said method comprising administering to said mammal an effective amount for treating said dysfunction a compound of compound of the formula 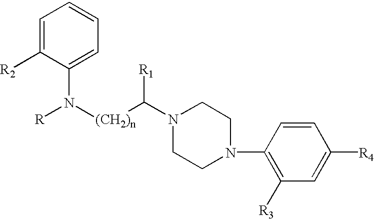
wherein
R=is a hydrogen atom, an alkylcarbonyl, a cycloalkylcarbonyl, a substituted cycloalkylcarbonyl or a monocyclic heteroarylcarbonyl group;
R₁ is chosen from the group consisting of hydrogen atom and lower alkyl group;
R₂ is chosen from the group consisting of halogen atom, alkoxy, phenoxy, nitro, cyano, acyl, amino, acylamino, alkylsulphonylamino, alkoxycarbonyl, N-acylaminocarbonyl, N-alkylaminocarbonyl, N,N-dialkylaminocarbonyl, aminocarbonyl, trifluoromethyl and polyfluroalkoxy groups;
R₃ is chosen from the group consisting of methoxy and polyhaloalkoxy;
R₄ is chosen from the group consisting of halogen, hydroxyl, lower alkoxy, lower acyloxy, lower N-alkylaminocarbonyloxy and lower N, N-alkylaminocarbonyloxy; and
n=1 or 2;
or an enantiomer, N-oxide, hydrate or pharmaceutically acceptable salt thereof.

24. The method of claim 23 wherein R is a cycloalkylcarbonyl group, R₂ is an alkoxy or trifluoroalkoxy group, R₃ is a polyhaloalkoxy group and R₄ is a halogen atom.

25. The method of claim 23 wherein R is a cycloalkylcarbonyl group, R₂ is a trifluoroalkoxy group, R₃ is a methoxy group and R₄ is selected from the group consisting of hydroxyl, lower alkoxy, lower acyloxy, lower N-alkylaminocarbonyloxy and lower N, N-dialkylaminocarbonyloxy groups.

26. The method of claim 23 wherein said administration is effective for ameliorating at least one of urinary urgency, increased urinary frequency, incontinence, urine leakage, enuresis, dysuria, urinary hesitancy, and difficulty in bladder emptying in said mammal.

27. The method of claim 26 wherein said mammal is a human.

28. The method of claim 23, wherein said administering is achieved using a route selected from the group consisting of oral, enteral, intravenous, intramuscular, subcutaneous, transmucosal, transdermal, and by-inhalation routes.

29. The method of claim 23, wherein said compound is administered to said mammal in an amount of between about 0.01 and 25 mg/kg/day.

30. The method of claim 23, wherein said compound is administered to said mammal in an amount of between about 0.2 and about 5 mg/kg/day.

31. The method of claim 23, wherein said compound is administered to said mammal in an amount of between about 50 and 400 mg/day.

32. The method of claim 28, wherein said administering is achieved using a route selected from the group consisting of oral and transdermal routes.

33. The method of claim 32, wherein the amount of said compound is between about 0.1 and 10 mg/kg/day.

34. A method for treating neuromuscular dysfunction of the lower urinary tract in a mammal in need of such treatment, said method comprising administering to said mammal an effective amount for treating said dysfunction a compound chosen from the group consisting
1-[N-cyclohexylcarbonyl-N-(2-methoxyphenyl)-2-aminoethyl]-4-[4-fluoro-2-(2,2,2-trifluoroethoxy)phenyl]piperazine;
1-[N-cyclohexylcarbonyl-N-(2-trifluoromethoxyphenyl)-2-aminoethyl]-4-[4-fluoro-2-(2,2,2-trifluoroethoxy)phenyl]piperazine;
1-[N-cyclohexylcarbonyl-N-(2-trifluoromethoxyphenyl)-2-aminoethyl]-4-(2,4-dimethoxyphenyl)piperazine;
1-[N-cyclohexylcarbonyl-N-(2-trifluoromethoxyphenyl)-2-aminoethyl]-4-(4-hydroxy-2-methoxyphenyl)piperazine;
1-[N-cyclohexylcarbonyl-N-(2-trifluoromethoxyphenyl)-2-aminoethyl]-4-(4-acetoxy-2-methoxyphenyl)piperazine;
1-[N-cyclohexylcarbonyl-N-(2-trifluoromethoxyphenyl)-2-aminoethyl]-4-(4-ethylaminocarbonyloxy-2-methoxyphenyl)piperazine; and
1-[N-cyclohexylcarbonyl-N-(2-trifluoromethoxyphenyl)-2-aminoethyl]-4-[4-(2-methylpropionyloxy)-2-methoxyphenyl]piperazine;
and enantiomers, N-oxides, hydrates, and pharmaceutically acceptable salts thereof.

35. The method of claim 34, wherein said administration is effective for ameliorating at least one of urinary urgency, increased urinary frequency, incontinence, urine leakage, entiresis, dysuria, urinary hesitance, and difficulty in bladder emptying in said mammal.

36. The method of claim 35 wherein said mammal is a human.

37. The method of claim 34, wherein said administering is achieved using a route selected from the group consisting of oral, enteral, intravenous, intramuscular, subcutaneous, transmucosal, transdermal, and by-inhalation routes.

38. The method of claim 34, wherein said compound is administered to said mammal in an amount of between about 0.01 and 25 mg/kg/day.

39. The method of claim 34, said compound is administered to said mammal in an amount of between about 0.2 and about 5 mg/kg/day.

40. The method of claim 34, wherein said compound is administered to said mammal in an amount of between about 50 and 400 mg/day.

41. The method of claim 34, wherein said administering is achieved using a route selected from the group consisting of oral and transdermal routes.

42. The method of claim 41, wherein the amount of said compound is between about 0.1 and 10 mg/kg/day.
